(19) United States
(12) Patent Application Publication
(10) Pub. No.: US 2026/0145033 A1
(43) Pub. Date: May 28, 2026

(54) SYSTEMS AND METHODS OF PROVISIONING A FITNESS RECOMMENDATION IN RELATION TO A USER

(71) Applicants: Yosenia Flores, Yonkers, NY (US); Edwin Briu, Yonkers, NY (US)

(72) Inventors: Yosenia Flores, Yonkers, NY (US); Edwin Briu, Yonkers, NY (US)

(21) Appl. No.: 19/178,478

(22) Filed: Apr. 14, 2025

Related U.S. Application Data
(60) Provisional application No. 63/633,484, filed on Apr. 12, 2024.
(60) Provisional application No. 63/712,273, filed on Oct. 25, 2024.

(51) Int. Cl. A63B 24/00; G16H 20/30; G16H 20/70

(52) U.S. Cl. CPC A63B 24/0075; G16H 20/30; G16H 20/70; A63B 2225/20

(57) ABSTRACT

The present disclosure provides a method of provisioning a fitness recommendation in relation to a user. Further, the method may include receiving, using a communication device, user physical-characteristic data from a user device associated with a user. Further, the user physical-characteristic data represents a physical characteristic associated with a user body associated with the user. Further, the method may include analyzing, using a processing device, the user physical-characteristic data. Further, the method may include generating, using the processing device, ideal-fitness recommendation data based on the analyzing. Further, the ideal-fitness recommendation data corresponds to a recommendation in relation to an ideal fitness associated with the user body. Further, the generating may be further based on an ideal fitness formula. Further, the method may include transmitting, using the communication device, the ideal-fitness recommendation data to the user device.

### FIELD OF DISCLOSURE

The present disclosure relates to the field of data processing. More specifically, the present disclosure relates to systems and methods of provisioning a fitness recommendation in relation to a user.

### BACKGROUND

The field of health and fitness is pivotal in promoting overall well-being and preventing chronic conditions associated with poor health practices. In today's fast-paced world, maintaining optimal body weight is crucial for enhancing physical performance, energy levels, and disease prevention. Personalized approaches to weight management have gained traction as individuals seek tailored solutions that cater to their unique characteristics.

The objective of this field is to provide individuals with accurate and adaptable tools to maintain a healthy body weight. This involves understanding the factors that influence weight, such as genetics, lifestyle, and environmental variables. The desire for a more personalized approach stems from the recognition that one-size-fits-all solutions often fail to meet individual needs effectively.

Existing systems may fall short due to several limitations. One common issue is the lack of customization, where weight management advice does not account for individual differences in muscle mass, genetics, or lifestyle. This can lead to dissatisfaction and poor adherence when users feel the recommendations are too generic. Additionally, many systems fail to provide real-time adjustments as factors like muscle gain or loss fluctuate over time, making their estimates less accurate and relevant.

Another challenge is the difficulty in maintaining consistent weight goals, often due to external influences such as stress or changing lifestyles. Without dynamic adjustments, existing methods may not keep pace with these changes, leading to ineffective results for users.

Therefore, improved systems that can offer personalized, adaptive, and practical solutions to facilitate optimal body weight management, addressing the gaps identified in current approaches are requires. Therefore, there is a need for improved systems and methods of provisioning a fitness recommendation in relation to a user.

### SUMMARY OF DISCLOSURE

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

The present disclosure provides a method of provisioning a fitness recommendation in relation to a user. Further, the method may include receiving, using a communication device, user physical-characteristic data from a user device associated with a user. Further, the user physical-characteristic data represents a physical characteristic associated with a user body associated with the user. Further, the method may include analyzing, using a processing device, the user physical-characteristic data. Further, the method may include generating, using the processing device, ideal-fitness recommendation data based on the analyzing. Further, the ideal-fitness recommendation data corresponds to a recommendation in relation to an ideal fitness associated with the user body. Further, the generating may be further based on an ideal fitness formula. Further, the method may include transmitting, using the communication device, the ideal-fitness recommendation data to the user device.

The present disclosure provides a system of provisioning a fitness recommendation in relation to a user. Further, the system may include a communication device. Further, the communication device may be configured for receiving user physical-characteristic data from a user device associated with a user. Further, the user physical-characteristic data represents a physical characteristic associated with a user body associated with the user. Further, the communication device may be configured for transmitting ideal-fitness recommendation data to the user device. Further, the system may include a processing device. Further, the processing device may be configured for analyzing the user physical-characteristic data. Further, the processing device may be configured for generating the ideal-fitness recommendation data based on the analyzing. Further, the ideal-fitness recommendation data corresponds to a recommendation in relation to an ideal fitness associated with the user body. Further, the generating may be further based on an ideal fitness formula.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTIONS OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform 100 consistent with various embodiments of the present disclosure.

FIG. 2 is a block diagram of a computing device 200 for implementing the methods disclosed herein, in accordance with some embodiments.

FIG. 3 illustrates a flowchart of a method 300 of provisioning a fitness recommendation in relation to a user, in accordance with some embodiments.

FIG. 4 illustrates a flowchart of a method 400 of provisioning a fitness recommendation in relation to a user including generating, using the processing device 504, a modified ideal-fitness recommendation data, in accordance with some embodiments.

FIG. 5 illustrates a block diagram of a system 500 of provisioning a fitness recommendation in relation to a user, in accordance with some embodiments.

FIG. 6 illustrates a user interface of an application associated with systems and methods of provisioning a fitness recommendation in relation to a user, in accordance with some embodiments.

FIG. 7 illustrates a user interface of an application associated with systems and methods of provisioning a fitness recommendation in relation to a user, in accordance with some embodiments.

FIG. 8 illustrates a user interface of an application associated with systems and methods of provisioning a fitness recommendation in relation to a user, in accordance with some embodiments.

FIG. 9 illustrates a user interface of an application associated with systems and methods of provisioning a fitness recommendation in relation to a user, in accordance with some embodiments.

FIG. 10 illustrates a user interface of an application associated with systems and methods of provisioning a fitness recommendation in relation to a user, in accordance with some embodiments.

FIG. 11 illustrates a user interface of an application associated with systems and methods of provisioning a fitness recommendation in relation to a user, in accordance with some embodiments.

### DETAILED DESCRIPTION OF DISCLOSURE

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of the disclosed use cases, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

### Overview:

The present disclosure describes methods and systems for facilitating provisioning of fitness recommendation data.

In some embodiments, the given disclosure describes a platform referred as “Briu Weight Tracker” application, which is a tool designed to help users track and manage their ideal weight. On the screen, two stylized human figures are presented: a male one on the left and a female on the right, which represent the gender options available to the user. Below the figures, there is the “Select Gender” option, which tells the user to choose their gender to customize the experience of using the application.

Further, the selection of the gender on this screen is a fundamental step for the precise calculation of the ideal weight using the Ideal Weight Formula (FPI). The formula adjusts the calculations based on the user's gender, since men and women have significant differences in terms of body composition, muscle mass, and fat distribution. For example, for men, specific coefficients are applied that reflect greater muscle mass and average bone density. Further, for women, adjustments are used that consider a higher natural percentage of body fat and hormonal differences.

Further, by selecting gender, the user activates the corresponding route within the FPI formula, ensuring that all subsequent calculations (such as the ideal weight, calories burned at rest, and the percentage of body fat) are precisely adapted to the specific physiological characteristics of the selected gender.

Further, after the user selects the gender on the screen, the application directs him to the next screen, where the user may be asked for other personal data necessary for the calculation of the ideal weight, such as height, current weight, and level of physical activity, among others. These data, together with the gender selection, allow the FPI formula to generate a personalized calculation of the ideal weight for the user.

Further, the interface is minimalist and easy to use, allowing the user to quickly start the customization process with a simple tap on the figure corresponding to their gender. This functionality ensures that all the calculations made by the application are relevant and accurate, specifically adapted to the biological needs of the user.

In some embodiments, the given disclosure describes a platform referred as “Briu Weight Tracker” application, where the user must select their body type or somatotype. The interface presents three stylized human figures, each representing one of the three main somatotypes. Further, the three main somatotypes may include ectomorph, mesomorph and endomorph. Further, the Ectomorph is characterized by a thin structure and low amount of muscle mass. Further, Mesomorph is characterized by a muscular and athletic constitution with a balance between muscle mass and fat. Further, Endomorphic reflects a greater tendency to accumulate fat with a rounder body structure.

Further, the selection of the somatotype is an essential component in the calculation of the ideal weight using the Ideal Weight Formula (FPI). The somatotype directly influences the way the body distributes muscle mass and fat, which significantly affects the results of the formula. For example, Ectomorphs usually have less muscle mass and a thinner structure, so the FPI formula adjusts the calculations to reflect a lower ideal weight and possibly a lower percentage of body fat. Further, Mesomorph may be defined as a somatotype with a balance between muscle mass and fat, the FPI formula applies adjustments that maintain the balance when calculating the ideal weight. Further, for endomorphs, who tend to accumulate more body fat, the FPI formula adjusts the calculations to reflect an ideal weight that considers this trend, with a focus on reducing excess fat. The selection of the somatotype ensures that the ideal weight formula is customized according to the specific physical characteristics of the user, providing a more accurate and relevant calculation.

Further, once the user selects the somatotype, the “Next” button is pressed by the user to advance to the next stage, where the user may be asked for more personal details, such as height, current weight and level of physical activity. These additional data, combined with the selected somatotype, allow the FPI formula to calculate a highly personalized ideal weight.

Further, the interface is intuitive, with clear images that facilitate the identification of the corresponding somatotype ensuring that the user may quickly and accurately select body type, which is essential for obtaining accurate results in the calculation of the ideal weight. The “Next” button provides a smooth navigation, guiding the user to the next step in the customization process.

In some embodiments, the given disclosure describes a platform referred as “Briu Weight Tracker” application where the user may enter the age. The interface is minimalist, with a clean design that shows a number in the center, in this case “46”, which represents the current age entered. On each side of the number there are increase and decrease buttons, represented by a “+” and a “−”, which allow the user to adjust their age up or down. At the bottom of the screen, a blue button labeled “Next” allows the user to proceed to the next step once the user has entered the age.

Further, age is a critical factor in the calculation of the ideal weight using the Ideal Weight Formula (IPF). The formula adjusts the ideal weight calculations according to the user's age, since body composition and metabolism change over time. As people get older, people tend to lose muscle mass and increase in body fat, which directly impacts the formula. Further, for young people an adjustment is applied that considers a faster metabolism and a greater amount of muscle mass. Further, for older adults, the formula adjusts the ideal weight to reflect a decrease in bone and muscle density, along with a slower metabolism. Further, by entering age on the screen, the user ensures that the calculations made by the FPI formula are accurate and specific to the user's stage of life, providing a result that better reflects the current physiological needs.

Further, once the user has entered their correct age using the increase or decrease buttons, the “Next” button is pressed to proceed to the next step in the customization process. The customization process could include the introduction of other factors such as height, current weight, or level of physical activity, all of which are combined with age to calculate the ideal weight.

Further, the interface is easy to use, allowing the user to adjust their age with simple taps on the buttons. The simplicity of the design minimizes the possibility of errors, ensuring that the data entered is accurate so that the application may make an exact calculation of the ideal weight based on the FPI formula.

In some embodiments, the given disclosure describes a platform referred as “Briu Weight Tracker” application where the user may enter height. The interface is simple and has a numerical value in the center of the screen, in this case “6′3″ (six feet and three inches), which represents the height entered. On each side of the value, there are increment and decrease buttons represented by a “+” and a “−”, which allow the user to adjust their height up or down. Below the height value, there are options to switch between the units of measurement: “CM” (centimeters) and “Feet” (feet), the “Feet” option is currently selected. At the bottom of the screen, a blue button labeled “Next” allows the user to proceed to the next step once he has entered his height.

Further, height is a fundamental factor in the calculation of the ideal weight using the Ideal Weight Formula (FPI). The user's height directly affects the amount of muscle mass and the fat distribution that is considered optimal for maintaining a healthy weight. In addition, the formula also takes into account the relationship between height and other factors, such as muscle mass and body composition, to provide a personalized and accurate calculation. Further, for example, Greater height, a higher person may require a higher caloric intake and an adjustment in optimal muscle mass, which may result in a higher ideal weight. Further, for lower height, a shorter person, the formula may adjust the caloric and muscle mass recommendations to reflect a lower ideal weight.

Further, when entering his height, the user allows the FPI formula to perform precise and personalized calculations, which not only consider height but also how it relates to muscle mass and body composition in general.

Further, once the user has entered their correct height using the increment or decrease buttons, press the “Next” button to proceed to the next step in the customization process, which could include the introduction of other factors such as the current weight, the level of physical activity, and the specific fitness goals.

Further, the interface is easy to use, allowing the user to adjust its height with simple taps on the buttons. The possibility of switching between different units of measurement (centimeters or feet) adds flexibility, adapting to the user's preferences. The minimalist design and the “Next” button make the process quick and simple.

In some embodiments, the given disclosure describes a platform referred as “Briu Weight Tracker” application where the user may select the main fitness objective. The interface offers several options, organized in a vertical list. Further, the list may include Weight Loss, Gain Weight, Maintain Weight, Increase Muscle Mass, and Weight Lose & Increase Muscle Mass.

Further, the user may select an option that best aligns with their personal health and fitness goals. Once selected, the blue button labeled “Next” at the bottom of the screen allows the user to proceed to the next step. The selection of an objective is essential to adapt the recommendations and calculations made by the Ideal Weight Formula (FPI) within the application. Each objective involves specific adjustments in the formula that affect how the ideal weight, the calories needed, and other related parameters are calculated. Further, for example, for Weight Loss, the formula is adjusted to create a caloric deficit that helps the user lose weight while maintaining muscle mass. Further, here, the formula is adjusted to calculate a caloric surplus necessary to gain weight in a healthy way. Further, the formula ensures a caloric balance to maintain the user's current weight. Further, adjustments are made to increase protein and calorie intake, supporting muscle growth. The selected option guides the application to customize the recommendations and calculations, ensuring that the user's goals are effectively achieved.

Further, after selecting the objective, the user presses “Next” to proceed to the next stage of the customization process in the application, where he may be asked to enter more details such as the level of physical activity, caloric intake, and other relevant factors.

Further, the interface is simple and direct, allowing the user to select the target with a simple touch. The “Next” button facilitates smooth navigation to the next steps, making the user experience efficient and clear.

In some embodiments, the given disclosure describes a platform referred as “Briu Weight Tracker” application where the user must select their daily meal plan. The interface presents three options organized in a list. Further, the list may include 3 meals a day, 4 meals a day and 5 meals a day.

Further, on selecting the “3 Meals per Day” option the user may choose one of the other two options if the user prefers. Once the user has made his decision, the user may click on the blue button labeled “Next” at the bottom of the screen to proceed to the next step.

Further, the selection of the number of meals per day is crucial for the proper distribution of macronutrients calculated using the Ideal Weight Formula (IPF). Depending on the number of meals selected, the application may adjust the amount of protein, carbohydrates and fats that the user may consume with each meal to achieve their fitness goal, whether it is weight loss, muscle mass gain or weight maintenance. Further, for example, for 3 meals a day, the formula may divide the calculated daily macronutrients into three equal parts, optimizing each meal to meet the user's goals. Further, for 4 meals a day, macronutrients may be distributed in four meals, providing a constant flow of nutrients throughout the day. Further, for five meals a day, those who prefer to eat more frequently, macronutrients may be adjusted in five portions, maintaining a regular supply of energy and nutrients.

Further, the selection of the meal plan allows additional customization, ensuring that the user receives precise recommendations that align with their lifestyle and dietary preferences.

Further, after selecting their meal plan, the user presses “Next” to continue with the customization of the profile within the application. This may include the introduction of more details related to eating habits, preferences, and other factors that affect the nutrition plan and the calculation of the ideal weight.

Further, the interface is simple and direct, allowing the user to select their preferred meal plan with ease. The arrangement of the options and the “Next” button make the process efficient and easy to follow, ensuring that the user may quickly advance to the next step.

In some embodiments, the given disclosure describes a platform referred as “Briu Weight Tracker” application where the user must select the frequency of their weekly exercise activity. The interface presents five options, organized in a vertical list.

Further, the list may include I don't work out, I work out 1-2 times per week, I work out 5-6 times per week, and I work out 7 times per week.

Further, the user may choose an option that best represents their usual level of exercise. Once the option is selected, the user may proceed to the next step by pressing the blue button labeled “Next” at the bottom of the screen.

Further, the selection of the level of exercise activity is a determining factor in the calculation of the ideal weight and the calories burned using the Ideal Weight Formula (FPI). The frequency of exercise influences the amount of calories that must be consumed to reach and maintain the ideal weight, as well as the adjustment of muscle mass and other relevant parameters. The formula also takes into account the user's muscle mass, which allows a more precise customization depending on the level of exercise. Further, in I don't Work out, the formula adjusts the necessary calories down to reflect a sedentary lifestyle, with a minimal impact on muscle mass. Further, in I work out 1-2 times per week, it is considered a mild physical activity, with a minimum adjustment in the necessary calories and a moderate focus on the preservation of muscle mass. Further, in I work out 3-4 times per week, the formula increases calories to reflect a moderate level of exercise and adequate maintenance of muscle mass. Further, in I work out 5-6 times per week, with a high level of exercise, the formula calculates a significant increase in the calories needed to maintain energy balance and optimize the gain or maintenance of muscle mass. Further, in I work out 7 times per week, for those who exercise daily, the formula maximizes caloric intake and adjustments in muscle mass to support the level of extreme activity and optimize body composition.

Further, the selection of the frequency of exercise allows the FPI formula to adapt to the user's exercise habits, providing personalized and precise recommendations both in caloric intake and in the preservation and improvement of muscle mass.

Further, after selecting the exercise frequency, the user presses “Next” to continue with the customization of his profile within the application. This may include the entry of other personal data, such as current weight, caloric intake, and fitness goals.

Further, the interface is simple and easy to use, allowing the user to select their level of exercise activity with a simple touch. The clear design and the “Next” button facilitate smooth navigation, ensuring that the user may quickly advance in the customization process.

In some embodiments, the given disclosure describes a platform referred as “Briu Weight Tracker” application, which offers a complete breakdown of the user's fitness goals and personalized recommendations based on their data. The screen is divided into several sections that detail the caloric intake, the distribution of macronutrients, the factors that affect the ideal weight, and graphs that visualize the user's progress.

Further, the daily intake of Macronutrients may include Carbohydrates Intake (455.14 g): 1820.58 cal, Protein Intake (182.06 g): 728.23 cal, Fat Intake (121.37 g): 1092.35 cal, Daily Caloric Intake: 3641.16 cal, Food Distribution by Food: Further, the food distribution by food may include Carbohydrates (152.00 g): 608.00 cal, Proteins (61.00 g): 244.00 cal, Fats (40.00 g): 360.00 cal, and Each Meal: 1213.72 cal. Further, the factors that affect the Ideal Weight may include Medical Conditions by 10%, Level of Hydration by 5%, and Perceived Stress Level by 5% to 10%. Further, the Progress Charts may include Current Weight: 220.2 lb., You under Weight by: 4.8 lb, Your Body Fat % is under: −2.2%, Ideal Weight: 225.0 lb, and Ideal Body Fat %: 10.5%. Further, the calories may include Burn at Rest: 2936.4 cal, Daily Calories Intake: 3641.2 cal, and Daily Water Intake: 3.6 Liter.

Further, the screen provides a complete view of how the Ideal Weight Formula (FPI) has customized fitness and nutrition recommendations for the user. Daily calories, the distribution of macronutrients, and other factors have been calculated taking into account height, weight, muscle mass, level of physical activity, and the specific objectives of the user. The formula also considers factors that may influence the ideal weight, such as medical conditions, hydration, and stress levels, providing a comprehensive strategy to reach and maintain the ideal weight.

Further, the interface is informative and easy to interpret, allowing the user to see a clear summary of their goals and the progress towards them. The “Finish” button facilitates the completion of the process, marking the closing of the custom configuration and allowing the user to start implementing the recommendations provided.

In some embodiments, the given disclosure describes a platform referred as “Briu Fitness App” (or “Briu Fitness Studio App”) application. The interface is designed to collect basic user information, such as height, current weight, target weight, and calories you need to burn, to customize the results and optimize the performance of the fitness program.

Further, the sections and details may include subtext. Further, the subtext may include “Let us know you better to help you boost your workout results.” Further, the Body Measurement Fields may include Height. Further, the height shows “6 ft. 3 in” (6 feet and 3 inches), allowing the user to enter its height. Further, the Current weight shows “210.0 lbs.” (210.0 pounds), allowing the user to enter their current weight. The Target weight shows “225.0 lbs.” (225.0 pounds), which is the weight that the user wants to reach. The Weight Need to loss may include Sample “15.0 lbs.” (15.0 pounds), automatically calculated based on the current weight and the target. The Calories Need to Burn shows “52,500.00”, which are the calories needed to lose the desired weight, calculated according to the FPI and FCLP formulas. Further, the Adjustment Controls may include Buttons to Adjust the Target Weight which includes “−” and “+” buttons to decrease or increase the target weight, with the current value shown in the center (225.0 lbs). Further, the “Save & Continue” button” allows the user to save the information entered and continue with the process of customization of the fitness plan.

Further, the screen is crucial in the “Briu Fitness App” (or “Briu Fitness Studio App”) because it uses the FPI, FCLP, FCMP and the Briu Method, formulas to calculate the ideal target weight and the calories needed to reach it. The FPI formula helps determine the appropriate target weight, the FCLP calculates how many calories must be burned to reach that weight, and the FCMP may influence the management of muscle mass during the process.

Further, the body measurement entry screen allows the user to customize their fitness plan by entering key data about their body. This information is used to calculate precise weight and calorie goals, which helps to create a more effective and results-oriented training plan.

In some embodiments, the given disclosure describes a platform referred as “Briu Fitness App” (or “Briu Fitness Studio App”) application, where the user may manage and follow their weekly training program. The interface is designed to guide the user through a fitness plan structured by weeks, with details about training sessions and rest days.

Further, the sections and details may include a welcome message. Further, the welcome message may include a text and a subtext. Further, the text may include “Welcome to Briu Fitness Studio”. Further, the subtext may include “Our mission is to help you achieve your fitness goal, and get you into perfect shape. ‘Fitness is 100% mental. Your body won't go where your mind doesn't push it.’” Further, the management of the training week may include a headline and listed weeks. Further, the headline may include “Start your week”. Further, the listed weeks may include Week 1, Week 2, Week 3, and Week 4. Further, the Week 1 may include that it includes 52 weeks with 45 minutes of training each. There is a progress indicator with gray circles for workouts and yellow circles for rest days. The “Start” button allows you to start week 1. Further, similar to week 1, but the Week 2 button has a lock icon, indicating that it is not yet unlocked. Further, the week 3 may be configured the same as week 2, with a locked button. Further, the week 4 may be configured the same as the previous weeks, with a locked button.

Further, the screen is directly related to the personalized recommendations derived from the FPI, FCLP, FCMP and The Briu Method Formulas. The “Briu Fitness App” (or “Briu Fitness Studio App”) uses these calculations to structure a training plan adapted to the user's objectives, such as the optimization of the ideal weight, the burning of calories, and progressive muscle growth.

Further, the screenshot as shown in FIG. 17 comprises the drop-down options menu in the “Briu Fitness App” (or “Briu Fitness Studio App”) application. The menu is designed to provide quick access to several essential functions of the application, allowing the user to easily navigate between different sections that support their fitness program.

Further, the sections and details may further include an image where a muscular athlete appears at the top of the screen, emphasizing the dedication and effort required to achieve fitness goals. Further, the drop down menu options may include icons. Further, the Scale Icon may be represented by a yellow icon, probably allowing the user to track their weight or enter body measurements. Further, Water Drop Icon, represented by a blue icon, possibly related to the tracking of water intake or hydration. Further, Weights Icon, represented by a green icon, it indicates access to weightlifting workouts or routines. Further, People Group cone represented by a light blue icon could be related to the community, social networks or support groups within the application. Further, Apple and Tape Measure Icon, represented by an orange icon, suggests access to nutrition plans or monitoring caloric intake.

Further, the management of the Training Week may include Headline and listed weeks. Further, the headline may include “Start your week”. Further, Listed Weeks including Week 1 may include The “Start” button is available to start week 1. Further, in. Week 2 and Following Weeks: The buttons are locked, indicating that they have not yet been unlocked.

Further, the Bottom Navigation Menu may include Icons. Further, the Network, represented by an icon on the left. Further, the Exit, represented by a “+” icon in the center, possibly to close the menu or the application. Further, the Setting represented by a gear icon on the right is to access the application settings.

Further, the menu provides quick access to functions that may be directly related to the FPI, FCLP, FCMP and The Briu Method formulas used in the “Briu Fitness App” (or “Briu Fitness Studio App”). For example, weight tracking, caloric intake, and training plans are aligned with personalized recommendations derived from these formulas, which allows the user to effectively manage and optimize their fitness routine.

Further, the screenshot as shown in FIG. 18 may include the summary screen in the “Briu Fitness App” application (or “Briu Fitness Studio App”), where the user may view a summary of their progress in the fitness program. The interface is designed to provide an overview of the calories burned, the current weight, the target weight and the overall progress in terms of weight loss.

Further, the headline may include text and side icons. Further, the text may include a “Summary”, Further, the side icons may include exit icon and the editing icon. Further, the exit icon may be on the left, probably to return to the previous screen. Further, the editing icon may be on the right, possibly to edit the information shown.

Further, the calories may include Workout Done: Show “0/260”, indicating the number of completed workouts out of a total of 260. Further, Total Calories: Show “0.00”, which represents the total calories burned. Further, Remaining Cal shows “−52,500.00”, which are the remaining calories to be burned to reach the goal. Further, the weight may include Current Weight showing “210.0 lbs”, which is the current weight of the user. Further, the Remaining Weight shows “15.0 lbs”, which is the weight that remains to reach the target weight. Further, the Target Weight shows “225.0 lbs”, which is the established target weight. Further, the progress graphs may include a headline and a graphic. Further, the Headline illustrates Sample “0.00 Pounds you losing”. Further, the Graphic may include a bar chart that shows the progress in weight loss over the weeks. Currently, only the first week is shown with a bar for “210.0 lbs”.

Further, this screen is closely related to the FPI, FCLP, FCMP and the Briu Method formulas used in the “Briu Fitness App” (or “Briu Fitness Studio App”). The summary of calories burned, current weight, and target weight reflects the user's progress according to the personalized recommendations derived from these formulas. The FPI helps establish the ideal target weight, the FCLP calculates the calories needed to achieve that goal, and the FCMP monitors muscle growth and weight loss over time.

Further, the screenshot as shown in FIG. 19 may include the water intake tracking screen in the “Briu Fitness App” (or “Briu Fitness Studio App”) application. The interface is designed to help the user monitor their daily and annual water consumption, setting goals that promote adequate hydration as part of their fitness routine.

Further, the monitoring of water intake, although not directly related to the FPI, FCLP and FCMP formulas, is an important complementary aspect within the framework of the Briu Fitness App. Maintaining adequate hydration is essential to optimize physical performance, improve calorie burning and support muscle growth, all of which is monitored and adjusted through the main formulas of the application

Further, the water tracking screen makes it easy for the user to monitor their daily and annual water intake, promoting healthy habits and supporting their fitness goals. Users may easily record their consumption using the buttons to add or subtract water bottles, while the progress indicators visually show how they advance towards their goals.

In some embodiments, the given disclosure describes a platform referred as “Briu Fitness App” (or “Briu Fitness Studio App”) may include the “Muscles Report” screen. The interface is designed to provide the user with a detailed analysis of the different muscle areas worked on during the workouts. This screen allows users to visualize and monitor the progress of their muscle development throughout their fitness program.

Further, this screen is directly related to the FCMP (Progressive Muscle Growth Formula), which is used in the “Briu Fitness App” (or “Briu Fitness Studio App”) to monitor and adjust the user's muscle development. Users may see which muscle groups are developing and adjust their exercise routines accordingly, ensuring balanced and efficient progress throughout the body.

Further, the muscle report screen allows users to track the development of their muscles in detail, giving them the ability to identify areas that need more work. This information is crucial for those who seek balanced muscle development and want to optimize their physical performance using personalized recommendations derived from the FPI, FCLP, and FCMP formulas.

In some embodiments, the given disclosure describes a platform referred as “Briu Fitness App” (or “Briu Fitness Studio App”) application may include may include the “Muscles Performance” screen. The interface is designed to provide a detailed analysis of the user's muscle performance, allowing them to see the progress in terms of muscle gain, calorie burning, and fat loss.

Further, this screen is closely related to the formulas FCMP (Progressive Muscle Growth Formula) and FCLP (Calorie Formula for Weightlifting). The FCLP calculates the calories needed to achieve the fitness goals, while the FCMP monitors muscle growth and fat loss. This data is reflected in the overall performance of the user shown on this screen.

Further, the muscle performance screen provides a detailed view of the user's progress in terms of muscle development, calorie burning, and fat loss. This information allows the user to adjust their training program effectively to maximize the results, using the personalized recommendations derived from the FPI, FCLP, and FCMP formulas.

In some embodiments, the given disclosure describes a platform referred as “Briu Fitness App” (or “Briu Fitness Studio App”) application may include may include the “Week Performance” screen. The interface is designed to provide a visual and detailed summary of the user's physical performance over a week of training. This screen allows the user to evaluate their weekly progress in terms of completed workouts, calories burned, repetitions performed and muscle gain.

Further, this screen uses the FCLP (Calorie Formula for Weightlifting) to calculate the calories burned during training sessions, as well as the FCMP (Progressive Muscle Growth Formula) to monitor muscle gain. The information presented here helps the user adjust their training regime to maximize the effectiveness of their fitness program, based on personalized data.

Further, the weekly performance screen is a valuable tool for users to keep a constant track of their progress. It allows them to analyze how effective their workouts have been during the week and adjust their objectives according to the results obtained. This information, derived from the FPI, FCLP, and FCMP formulas, is crucial for any user who seeks to improve their physical performance and achieve their fitness goals.

Further, the screen may include the “Select Muscle Group” screen in the “Briu Fitness App” (or “Briu Fitness Studio App”) application. The interface is designed for the user to select the muscle groups they want to focus on during their training session.

Further, the screen is essential to customize the user's exercise routine, allowing him to concentrate on specific areas of his body that he wants to improve. The selection of muscle groups may guide the type of exercises that the application may recommend in subsequent sessions. This function is useful to create a specific and effective training plan, suitable for the user's individual objectives, whether it is to increase muscle mass, tone, or improve strength in specific areas of the body.

In some embodiments, the given disclosure describes a platform referred as “Briu Fitness App” (or “Briu Fitness Studio App”) application may include may include the “Select a workout” screen. This interface allows the user to select between different specific exercises that they want to perform during their training session.

Further, the screen is crucial to customize the user's training, allowing him to choose the specific exercises that best suit his fitness objectives. Whether the user seeks to develop strength in specific areas or improve muscle endurance, this selection ensures that the exercise routine is aligned with their personal goals

In some embodiments, the given disclosure describes a platform referred as “Briu Fitness App” (or “Briu Fitness Studio App”) application may include may include the “Military Press” exercise screen. On this screen, the user may set the amount of weight he wants to lift during the military press exercise.

Further, the screen allows users to customize their training session, adjusting the amount of weight they may lift in the military press exercise. The visualization of the exercise provides a clear guide for the correct form, helping the user to perform the exercise safely and effectively

Further, the screen may include a detailed report of the “Military Press” exercise within the “Briu Fitness App” application. On this screen, the user may see a summary of the repetitions performed, the weight used, the calories burned, and the progress in muscle gain.

Further, The Briu Fitness App, Briu Fitness Studio App or Briu Weight Tracker is an innovative and personalized fitness application designed to help users achieve their health and wellness goals efficiently. This application is based on several exclusive formulas and methods developed by Edwin Briu, which allow the user to adapt the training to the physical characteristics and individual capabilities of each user.

Further, the screen offers a clear summary of the user's performance in the military press exercise. It allows the user to track their progress over time, monitor their calorie burning, the weight used and muscle gain. The visualization of the weekly progress allows the user to observe their progress and adjust their training accordingly.

In some embodiments, the given disclosure describes a platform referred as “Briu Fitness App” (or “Briu Fitness Studio App”) application may include may include the “Chest Press” exercise screen. On this screen, the user begins to perform the exercise, and the application's algorithm determines the weight with which the training should begin.

Further, the Algorithm of the Briu Method determines the initial weight based on the Briu Method, starting with approximations before reaching the real weight. In this case, the user must start with 60 pounds for a series of 5 repetitions, then increase to 80 pounds for another series of 5 repetitions, and finally reach the real weight of 100 pounds for three sets of 5 repetitions. The first 60 and 80 pounds are approximate weights, while the actual weight, which represents the user's capacity, is 100 pounds. The Briu Method is used to dynamically adjust the training parameters based on the weight capacity entered by the user for each exercise. Training tracking records and stores exhaustive data for the analysis of user progress. The Briu Method is based on a consistent pattern of 5 repetitions per pound raised, regardless of the exercise performed. For example, if the user lifts 50 pounds, the algorithm determines that he must start with 30 pounds for a series of 5 repetitions, then 40 pounds for another series of 5 repetitions, and finally 50 pounds for three sets of 5 repetitions. This pattern of repetitions and series remains constant for all the numbers entered by the user, ensuring uniformity in different exercises. In addition, the Briu Method incorporates full-body workouts on a daily basis, targeting all muscle groups.

Further, the screen is crucial for the user to start performing the exercise. The Briu Method algorithm automatically adjusts the weight with which the user must start, providing a safe and effective progression in training. The number selected on the screen indicates the weight that the user is lifting at that time, which allows accurate tracking of their progress during the exercise session.

Further, the screen may include the interface during the performance of the “Chest Press” exercise in the “Briu Fitness App” application (or “Briu Fitness Studio App”). This is where the user carries out the last series of the exercise with his target weight, which in this case is 100 pounds.

Further, the Algorithm of the Briu Method may be for the Weight Selection Process: According to the Briu Method, the user begins with approximate weights before reaching his actual weight. In this case, the user started with 60 pounds for a series of 5 repetitions, then increased to 80 pounds for another series of 5 repetitions, and finally reached the actual weight of 100 pounds for the last series of 5 repetitions. This pattern ensures a safe and effective progression during training.

Further, this screen as shown is essential for the user to finish the “Chest Press” exercise with the target weight determined by the Briu Method algorithm. The algorithm ensures that the user follows a logical and safe progression in their workouts, starting with approximation weights before reaching their maximum weight. This not only helps prevent injuries, but also optimizes the user's muscle growth and performance.

Further, the main components may include FPI. Further, The Ideal Weight Formula (IFP) is used to calculate an individual's optimal body weight based on several factors, including height, muscle mass, somatotype, age, lifestyle, stress levels, hydration, medical conditions and climate. This formula offers an accurate and personalized estimate of the user's ideal weight, which is essential to establish realistic and achievable goals in their fitness program. By incorporating adjustments for muscle mass, somatotype and other factors, IPF guarantees that users may aim for an ideal weight that accurately reflects their body composition and health needs. Further, the main component may include FCLP. Further, the Calorie Formula for Weightlifting (FCLP) provides a detailed estimate of the calories burned during weightlifting activities. This formula takes into account the intensity of the exercise, the lifted weight, the number of repetitions, the duration of each repetition and an adjustable intensity factor. By offering an accurate and personalized estimate of caloric expenditure, the FCLP helps users optimize their training programs and nutrition plans, aligning them with specific objectives such as weight loss, increased muscle mass or maintenance. Further, the Progressive Muscle Growth Formula (FCMP) is designed to calculate the parameters necessary for progressive muscle growth. This formula incorporates factors such as the frequency of exercise, muscle mass and the intensity of training, ensuring that users may increase their muscle mass safely and effectively. Following the guidelines of the FCMP, users may maximize their muscle growth potential while avoiding stagnation and overtraining.

Further, the Briu Method is a central component of the Briu Fitness App or Briu Fitness Studio App offering a unique algorithm that dynamically adjusts the training parameters based on the data entered by the user. This method is based on a consistent pattern of 5 repetitions per pound raised, regardless of the exercise performed. For example, if the user lifts 100 pounds, the algorithm may recommend starting with 60 pounds for a series of 5 repetitions, then increasing to 80 pounds for another series of 5 repetitions, and finally to 100 pounds for three sets of 5 repetitions. This progression is designed to ensure that users achieve optimal gains in strength and muscle development through structured and systematic training.

Further, the application interface allows users to select their current lifting weight, and the algorithm automatically adjusts the training plan according to the Briu Method. This guarantees that users always train at the right level of intensity, promoting continuous improvement without the risk of overtraining or injuries. In addition, the algorithm incorporates daily full-body workouts, aimed at all muscle groups, to provide a complete and balanced training regimen.

Further, The Briu Method algorithm is designed to determine the optimal starting weight before the user reaches his actual weight, regardless of the maximum capacity of the user. This approach guarantees a progressive adaptation to the effort, starting with more manageable weights to prepare the body safely and efficiently.

Further, regardless of whether the user lifts 100 pounds or 300 pounds as their maximum weight, the algorithm may always automatically calculate the appropriate initial weight. For example, if the user enters that his target weight is 100 pounds, the algorithm may determine that he must start with an approximate weight of 60 pounds, then go to 80 pounds, and finally reach 100 pounds in the last phase. This pattern adapts dynamically according to the weight entered, ensuring adequate progression and optimal warm-up.

Further, this process is key to avoiding injuries, improving the technique and maximizing performance. The algorithm not only considers the final weight that the user wants to lift, but also the importance of a progressive preparation before reaching that weight. It is an approach that remains constant, regardless of whether the final weight is relatively low or very high, offering a personalized guide for each training session.

Further, the “Briu Fitness App” (or “Briu Fitness Studio App”) and “Briu Weight Tracker” tracks and stores complete data of each user, including the weight lifted, the repetitions completed, the calories burned and the percentages of muscle gain. This data is analyzed to provide users with a detailed view of their progress, helping them adjust their training plans as needed to stay on the path to their fitness goals. The intuitive interface of the application shows this information in an easy-to-understand way, allowing users to monitor their progress over time.

Further, the “Briu Fitness App” (or “Briu Fitness Studio App”) and “Briu Weight Tracker” combines the power of exclusive formulas (FPI, FCLP, FCMP) with the innovative Briu Method to offer a personalized and effective fitness solution. By dynamically adjusting training parameters based on user data and providing a detailed analysis of progress, the application empowers users to achieve their fitness goals efficiently and safely.

Further, the Differentiation of Traditional Formulas and Added Value of Invention may include advanced personalization based on individual factors, real time adaptation and dynamic adjustments, detailed and precise calculation of caloric expenditure on weightlifting, integration into an interactive digital platform, and simplicity and accessibility in the user experience. Further, the formulas developed in this invention (FPI, FCMP, and FCLP) offer a level of customization that significantly exceeds the traditional formulas used to estimate ideal weight, muscle growth and caloric expenditure. Unlike traditional methods that are mainly based on generic values such as BMI (Body Mass Index) or standard formulas that do not consider individual variability, these formulas allow specific adjustments based on Gender of the user, Somatotype (ectomorph, mesomorph, endomorph), Age and height, Level of physical activity, and Specific objectives of the user (weight loss, muscle mass gain, weight maintenance, etc.) Further, the customization allows the calculations to be much more precise and adapted to the unique characteristics of each individual, something that is not achieved with conventional methods.

Further, The FCMP (Progressive Muscle Growth Formula) introduces the ability to adjust muscle growth calculations based on the intensity of the training (measured in MET values) and the weekly weight progression. Traditional methods do not consider these dynamic changes and tend to use static estimates that do not fit the actual progress of the user. The inclusion of an adjustable “proportionality factor”, which is modified according to the MET, provides a much more realistic and adaptable approach.

Further, The FCLP (Calorie Formula for Weightlifting) adds a level of precision that standard formulas do not reach by including an adjustable “intensity factor”. This factor reflects the variations in the technique, rhythm and individual effort of each user, allowing a more accurate estimate of caloric expenditure. While traditional formulas are based only on the total exercise time or on general calculations, the FCLP considers Lifted weight (in kilograms), Number of repetitions, and Duration of each repetition, and Adjustable intensity according to individual effort.

Further, one of the biggest differences of this invention is the integration of the formulas in a digital application that not only guides the user step by step, but also adjusts the recommendations in real time according to the information entered. This continuous and automatic customization is not common in current solutions, which usually require manual calculations or static configurations. The application offers intuitive interfaces for the selection of gender, somatotype and other parameters, automated recommendations based on user objectives and data and real-time adjustments as the user progresses in their routine.

Further, unlike traditional formulas that require technical knowledge and manual calculations, the application developed in this invention simplifies the process of monitoring and adjusting health and physical performance parameters. The ease of use and fluid user experience make this solution accessible to a wide range of people, from beginners to advanced athletes.

Further, the invention overcomes the limitations of conventional methods by combining advanced customization, real-time dynamic adaptation, precision in the calculation of key variables, and efficient integration into a digital platform. These improvements not only offer greater value to users, but also demonstrate the novelty and inventiveness of the solution compared to existing alternatives.

Further, The Calorie Formula for Weightlifting (FCLP) is a unique tool developed to offer an accurate estimate of caloric expenditure during weightlifting activities. This formula considers a variety of factors that influence energy expenditure, including exercise intensity (measured in MET), lifted weight, the number of repetitions, the duration of each repetition and an adjustable intensity factor. The combination of these elements allows a detailed and personalized estimate of the calories burned during resistance training.

Further, the precise calculation of the caloric expenditure is crucial for the following. Further, Training Optimization help design effective and personalized training programs. Further, Weight Control contributes to the management of body weight through an adequate balance of calories. Further, Monitoring of Progress evaluates the progress of the training and make necessary adjustments. Further, Performance Improvement: identifies areas for improvement and maximizing physical performance. Further, Health and Well-being maintains a balance between physical activity and caloric intake for a healthy life.

Further, The FCLP uses the following formula: Total Calories (FCLP)=(MET×Weight in pounds'×0.453592×Number of Repetitions×0.0175×Duration of Repetitions in Seconds×Intensity Factor)/60.

Further, the components of the formula may include the following components. Further, MET (Metabolic Equivalent of Task) is the intensity of the exercise. Further, weight in pounds converted to kilograms facilitates a standardized comparison. Further, number of repetitions may include total number of repetitions performed. Further, duration of the repetition in seconds may include specific time of each repetition. Further, Oxygen factor (0.0175) may include adjustment for oxygen consumption. Further, Conversion to minutes stabilizes the caloric output. Further, Intensity Factor adjusts the calculation according to the intensity of the exercise (range: 1.0 to 1.5).

Further, personal trainers are for designing the exercise physiologists. Further, the exercise physiologists are for studies and research on energy expenditure. Further, specialists in physical conditioning for improving the conditioning programs. Further, Athletes and Athletes optimize the performance and achieve fitness goals. Further, fitness enthusiasts better understand the caloric expenditure and manage the weight effectively.

Further, The Intensity Factor is an adjustable component in the Calorie Formula for Weightlifting (FCLP) that reflects the variability in the intensity of exercise and other individual factors that may affect caloric expenditure. This factor allows the user to customize the formula to adapt to different levels of effort and specific training conditions.

Further, The Intensity Factor is crucial because the caloric expenditure not only depends on the time and amount of work done, but also on the intensity with which the exercise is carried out. Different people may burn different amounts of calories by doing the same activity due to variations in their technique, rhythm, and perceived effort.

Further, The Intensity Factor may typically vary between 1.0 and 1.5. Further, 1.0 (Low Intensity): Represents a low effort or a slower pace. It is appropriate for exercises where the lifting is done in a controlled way and with less weight. Further, 1.1 to 1.3 (Moderate Intensity): Indicates a moderate effort. It is suitable for most weightlifting sessions with a constant rhythm and a moderate load. Further, 1.4 to 1.5 (High Intensity): It reflects a high effort, with fast lifts or with a lot of weight. It is used in high-intensity sessions where it is sought to maximize the effort in each repetition.

Further, parameters of an Example: MET: 8, Weight in pounds: 20 lb, Number of repetitions: 15, Duration of the repetition in seconds: 4, Intensity Factors: 1.0 (low intensity), 1.2 (moderate intensity), 1.5 (high intensity) Calculation of Total Calories:

$$Low Intensity ( 1. ) :$$

$$Further , Total Calories ( FCLP ) = ( 8 \times 20 \times 0.453592 \times 15 \times 0.0175 \times 4 \times 1. ) / 60 = 1.2700576$$

$$Total Calories = 1.2700576$$

$$Moderate Intensity ( 1.2 ) :$$

$$Total Calories ( FCLP ) = ( 8 \times 20 \times 0.453592 \times 15 \times 0.0175 \times 4 \times 1.2 ) / 60 = 1.52406912$$

$$Total Calories = 1.52406912$$

$$High Intensity ( 1.5 ) :$$

$$Total Calories ( FCLP ) = ( 8 \times 20 \times 0.453592 \times 15 \times 0.0175 \times 4 \times 1.5 ) / 60 = 1.9050864$$

$$Total Calories = 1.9050864$$

Further, The Intensity Factor is a crucial component to calculate the caloric expenditure during weightlifting. Using this factor, the user may adjust the training to obtain more accurate estimates of the calories burned, thus optimizing fitness and nutrition plan.

Further, The FCLP formula has been designed to provide an accurate estimate of caloric expenditure during weightlifting sessions, taking into account various factors that affect energy consumption. Each of these factors and how they are integrated into the formula.

Further, The Calorie Formula for Weightlifting (FCLP) is as follows: Total Calories (FCLP)=⋅(MET×Weight in pounds'×0.453592×Number of Repetitions×0.0175×Duration of Repetitions in Seconds×Intensity Factor)/60.

Further, breakdown of the components are as follows. Further, the MET is a measure that represents the intensity of a physical activity. A MET is equivalent to the energy expenditure at rest. For example, a MET of 8 indicates that the activity burns 8 times more calories per minute than the resting state. Further, the MET provides a base value of the intensity of the exercise.

Further, weight in pounds converted to kilograms is the weight raised during exercise is converted from pounds to kilograms to standardize the measurement. Conversion: 1 Pounds=0.453592 Kilograms. Further, it allows a standardized comparison of the lifted weight.

Further, number of repetitions is the total number of repetitions performed during the training session captures the total workload.

Further, duration of the repetition in seconds is the specific time it takes to complete a repetition. Further, it provides a detailed temporary approach to the effort.

Further, oxygen factor (0.0175) adjusts the calculation to take into account the consumption of oxygen during physical activity. Further, it adjusts the calculation of energy expenditure.

Further, conversion to minutes comprises the total result divided by 60 to convert seconds to minutes for stabilizing the caloric output.

Further, intensity factor is a new component that varies according to the intensity of the exercise and other individual factors. A value is assigned between 1.0 (low intensity) and 1.5 (high intensity). It Adjust the calculation according to the specific intensity of the exercise.

Further, suppose the user does a weightlifting session with the following parameters:
MET: 8
Weight in pounds: 10 lb
Number of repetitions: 15
Duration of the repetition in seconds: 4
Intensity Factor: 1.2 (moderately high intensity)
Let's calculate the total calories step by step.
Converting the weight into pounds to kilograms:
Weight in kg=10×0.453592=4.53592

Calculating the numerator of the formula:

$$Numerator = 8 \times 4.53592 \times 15 \times 0.0175 \times 4 \times 1.2$$

Performing the calculations step by step:

$$8 \times 4.53592 = 36.28736$$

$$36.28736 \times 15 = 544.3104$$

$$544.3104 \times 0.0175 = 9.525432$$

$$9.525432 \times 4 = 38.101728$$

$$38.101728 \times 1.2 = 45.7220736$$

Dividing the result by 60 to convert to minutes:

$$Total Calories ( FCLP ) = ( 45.7220736 ) / 60 = 0.76203456$$

Further, Using the FCLP, it is estimated that approximately 0.76 calories would be burned in this specific weightlifting session.

Further, The Calorie Formula for Weightlifting (FCLP) provides an accurate and detailed tool to calculate the caloric expenditure on resistance exercises. By disaggregating each component of the formula, they are integrated to offer a complete and personalized estimate of caloric expenditure. This formula is ideal for coaches, athletes and anyone interested in optimizing their training and nutrition plan.

Further, an example for a moderate weightlifting session is as follows:

### Parameters of the Example

MET: 8
Weight in pounds: 20 lb
Number of repetitions: 10
Duration of the repetition in seconds: 3
Intensity Factor: 1.2 (moderately high intensity)

### Step-by-Step Calculation:

Converting the weight into pounds to kilograms:

$$Weight in kg = 20 \times 0.453592 = 9.07184$$

Calculating the numerator of the formula:

$$Numerator = 8 \times 9.07184 \times 10 \times 0.0175 \times 3 \times 1.2$$

Performing the calculations step by step:

$$8 \times 9.07184 = 72.57472$$

$$72.57472 \times 10 = 725.7472$$

$$725.7472 \times 0.0175 = 12.701556$$

$$12.701556 \times 3 = 38.104668$$

$$38.104668 \times 1.2 = 45.7256016$$

Dividing the result by 60 to convert to minutes:

$$Total Calories ( FCLP ) = ( 45.7256016 ) / 60 = 0.76209336$$

Further, in the specific weightlifting session, approximately 0.76 calories would be burned.

Further, an example of Intense Weightlifting Session is as follows:

### Parameters of the Example

MET: 10
Weight in pounds: 30 lb
Number of repetitions: 12
Duration of the repetition in seconds: 5
Intensity Factor: 1.5 (High intensity)

### Step-by-Step Calculation:

Converting the weight into pounds to kilograms:

$$Weight in kg = 30 \times 0.453592 = 13.60776$$

Calculating the numerator of the formula:

$$Numerator = 10 \times 13.60776 \times 12 \times 0.0175 \times 5 \times 1.5$$

Performing the calculations step by step:

$$10 \times 13.60776 = 136.0776$$

$$136.0776 \times 12 = 1632.9312$$

$$1632.9312 \times 0.0175 = 28.07629$$

$$28.076296 \times 5 = 140.38148$$

$$140.38148 \times 1.5 = 210.57222$$

Dividing the result by 60 to convert to minutes:

$$Total Calories ( FCLP ) = ( 210.572 \; 22 ) / 60 = 3.509537$$

In this intense weightlifting session, approximately 3.51 calories would be burned.

Further, an example of Gentle Weightlifting Session is as follows:

### Parameters of the Example

MET: 6
Weight in pounds: 15 lb
Number of repetitions: 8
Duration of the repetition in seconds: 4
Intensity Factor: 1.0 (low intensity)

### Step-by-Step Calculation:

Convert the weight into pounds to kilograms:

$$Weight in kg = 15 \times 0.453592 = 6.80388$$

Calculate the numerator of the formula:

$$Numerator = 6 \times 6.80388 \times 8 \times 0.0175 \times 4 \times 1.$$

Perform the calculations step by step:

$$6 \times 6.80388 = 40.82328$$

$$40.82328 \times 8 = 326.58624$$

$$326.58624 \times 0.0175 = 5.7152592$$

$$5.7152592 \times 4 = 22.8610368$$

$$22.8610368 \times 1. = 22.8610368$$

Dividing the result by 60 to convert to minutes:

$$Total Calories ( FCLP ) = ( 22.86103 \; 68 ) / 60 = 0.38101728$$

Further, in this gentle weightlifting session, approximately 0.38 calories would be burned.

Further, these examples have demonstrated how the Calorie Formula for Weightlifting (FCLP) may be used to calculate the caloric expenditure during different weightlifting sessions. Each example has shown how to adjust the parameters according to the intensity and duration of the exercise to obtain an accurate estimate of the calories burned. This tool is invaluable for those who seek to optimize their training and achieve their fitness goals effectively.

Further, The FCLP integrates several key factors, including the MET (Metabolic Equivalent of Task), the raised weight converted to kilograms, the number of repetitions, the duration of each repetition, the oxygen factor and an adjustable intensity factor. This combination allows an accurate and detailed estimate of the calories burned during endurance training.

Further, accuracy in the calculation of caloric expenditure is essential for several aspects of training and nutrition:

Training Optimization: Helps design effective and personalized training programs.

Weight Control: Contributes to the management of body weight through an adequate calorie balance.

Monitoring of Progress: Allows the user to evaluate the progress of the training and make the necessary adjustments.

Performance Improvement: It helps to identify areas of improvement and maximize physical performance.

Health and Well-being: Maintains a balance between physical activity and caloric intake for a healthy life.

Further, these examples show how to adjust the parameters according to the intensity and duration of the exercise to obtain an accurate estimate of the calories burned.

Further, The Calorie Formula for Weightlifting (FCLP) is an invaluable tool for coaches, athletes and fitness enthusiasts. Its ability to offer detailed and personalized estimates of calorie expenditure allows users to optimize their training programs and nutrition plans effectively.

Further, the creation of this formula has been a significant step towards improving the accuracy in the calculation of caloric expenditure in weightlifting, providing an advanced and useful tool to achieve fitness goals.

Further, muscle growth is a complex process influenced by various factors, including genetics, nutrition, rest and, crucially, the training routine. The Progressive Muscle Growth Formula (FCMP) has been developed to provide a systematic and quantifiable way to estimate muscle growth based on weekly weight progression and training frequency. The key components of the FCMP are detailed below.

Further, to use the FCMP, the following initial parameters are established:
Training frequency: 5 days a week
Weekly progression: Increase of 5 pounds each week
Proportionality factor (K): 0.000001
Initial muscle mass (M_initial): 127.5 lb (70 kg)

These initial parameters serve as a basis for calculating the volume of training and weekly muscle growth.

Further, the daily training volume (V_{Dialy}) is calculated for each week ((n)) using the weights raised in the approach series and the series with real weight. The formula is as follows:

V_{Dialy,n}=(W_{1,n}×R₁)+(W_{2,n}×R₂)+ (W_{real,n}×Rᵣₑₐₗ×Sᵣₑₐₗ), where (W__{1,n}): Weight in the first series of approaches in the week (n)·(W__{2,n}): Weight in the second series of approximation in the week (n)·(W__{real,n}): Weight in the real weight series in the week (n)·(R_₁): Number of repetitions in the first series of approaches. (R_₂): Number of repetitions in the second approximation series. (R_ᵣₑₐₗ): Number of repetitions in the real weight series. (S_ᵣₑₐₗ): Number of real weight series. If in week 1 (n=1) the user lift 60 pounds in the first approximation series, 80 pounds in the second approximation series and 100 pounds in the series with real weight, the calculation would be: V_{Dialy,n}=(60 lb×5)+(80 lb×5)+(100 lb×5×3)

Further, the weekly training volume (V_Weekly) is calculated by multiplying the daily volume by the training frequency ((F)), which is the number of training days per week:

V_{Weekly, n}=V_{Dialy, n}×F, where (F) is Training frequency (days per week).

Further, for example, If the user trains 5 days a week and the daily volume of week 1 is 2,200 pounds, the weekly volume would be:

$${V}_{w e ekly , n} = 2 , 200 lb \times 5.$$

Further, the Weekly muscle growth (G_n) is estimated using the weekly training volume and the proportionality factor ((k)): G, n=K__{Adjusted}×V_{Weekly}, n, where (k) is a proportionality factor that relates the volume of training to muscle growth. Further, if the weekly volume in week 1 is 11,000 pounds, muscle growth would be: G, 1=0.000001×11,000.

Further, the total volume accumulated (Vtotal, accumulated) after (N) weeks is calculated by adding up the weekly volumes of each week:

$${V}_{total , accumulated} = \sum {V}_{weekly , n} .$$

Further, if the weekly volumes of the first three weeks are 11,000, 11,625 and 12,250 pounds respectively, the total accumulated volume would be:

$$VTotal , accumulated = 11 , 000 + 11 , 625 + 12 , 250$$

Further, the percentage of accumulated muscle mass increase (Percentage of increase__{Accumulated, n}) after (N) weeks is calculated by dividing the total accumulated muscle growth by the initial muscle mass and multiplying by 100. Percentage of increase__{Accumulated, n⋅}=0.034875/127.5)×100.

Further, The FCMP provides a structured framework for estimating muscle growth based on a progressive training routine. Each component of the formula contributes to a deeper understanding of how the volume of training affects muscle growth:

Daily Training Volume (V__{Daily}): Evaluate the daily effort based on the weights and repetitions used.

Weekly Training Volume (V__{Weekly, n}): Expand the daily effort to a weekly perspective.

Weekly Muscle Growth (G_ₙ): Translate the volume of training into muscle growth using a proportionality factor.

Total Accumulated Volume (V__{Total, accumulated}): Add up the weekly efforts to provide a global view of training over time.

Percentage of Increase in Muscle Mass (Percentage of increase__{Accumulated, n}): Calculate the impact of training in terms of percentage of muscle mass gained, offering a clear metric of progress.

By following this methodology, the user may obtain a quantifiable estimate of the progress in muscle growth based on the training routine and weight progression.

Further, to use the FCMP to calculate the accumulated training volume and the percentage of increase in muscle mass over several weeks, using specific data and a weekly weight progression.

### Initial Data

For this example, the following parameters may be used:
Training frequency: 5 days per week
Weekly progression: Increase of 5 pounds each week
Proportionality factor (K): 0.000001
Initial muscle mass (initial M_): 127.5 lb (70 kg)

### Week 1

### Weights Used:

First series of approach: 60 Pounds×5 Repetitions
Second series of approaches: 80 Pounds×5 Repetitions
Series with real weight: 100 Pounds×5 Repetitions×3 series

### Calculation of the Daily Volume:

$$VDialy , 1 = ( 6 lb \times 5 ) + ( 80 lb \times 5 ) + ( 100 lb \times 5 \times 3 )$$

$$VDialy , 1 = 300 + 400 + 1 , 500 VDialy , 1 = 2 , 200 lb$$

### Calculation of the Weekly Volume:

$${V}_{weekly , 1} = {V}_{Dialy , n 1} \times 5$$

$$Weekly , 1 = 2 , 200 lb . \times 5$$

$$Weekly , 1 = 11 , 000 lb .$$

### Weekly Muscle Growth Calculation:

$${G}_{1} = K \times {V}_{W e ekly , 1} {G}_{1} = 0.0 \; 00001 \times 11 , 000 {G}_{1} = 0.011 lb$$

### Week 2

### Weights Used:

First series of approach: 65 Pounds×5 Repetitions
Second series of approaches: 85 Pounds×5 Repetitions
Series with real weight: 105 Pounds×5 Repetitions×3 series

### Calculation of the Daily Volume:

$${V}_{Daily , 2} = ( 65 lb \times 5 ) + ( 85 lb \times 5 ) + ( 105 lb \times 5 \times 3 )$$

$${V}_{Daily , 2} = 325 + 425 + 1 , 575 {V}_{Daily , 2} = 2 , 325 lb$$

### Calculation of the Weekly Volume:

$${V}_{weekly , 2} = {V}_{Daily , 2} \times 5 {V}_{Weekly , 2} = 2 , 325 lb \times 5 {V}_{Weekly , 2} = 11 , 625 lb$$

### Weekly Muscle Growth Calculation:

$${G}_{2} = K \times {V}_{Weekly , 2} {G}_{2} = 0.0 \; 00001 \times 11 , 625 {G}_{2} = 0.0 \; 11625 lb$$

### Week 3

Weights Used:
First series of approach: 70 Pounds×5 Repetitions
Second series of approaches: 90 Pounds×5 repetitions
Series with real weight: 110 Pounds×5 Repetitions×3 series

### Calculation of the Daily Volume:

$${V}_{Dialy , 2} = ( 70 lb \times 5 ) + ( 90 lb \times 5 ) + ( 110 lb \times 5 \times 3 )$$

$${V}_{Dialy , 2} = 350 + 450 + 1 , 650 {V}_{Dialy , 2} = 2 , 450 lb$$

### Calculation of the Weekly Volume:

$${G}_{3} = {V}_{d ialy , 3} \times 5 {V}_{Weekly , 3} = 2 , 450 lb \times 5 {V}_{Weekly , 3} = 12 , 250 lb$$

### Weekly Muscle Growth Calculation:

$${G}_{3} = K \times {V}_{W e ekly , 3} {G}_{3} = 0.0 \; 00001 \times 12 , 250 {G}_{3} = 0.0 \; 1225 lb$$

Total Accumulated Volume and Percentage of Increase in Muscle Mass Total Accumulated Volume after 3 weeks:

$${V}_{Total , accumulated} = {V}_{Weekly , 1} + {V}_{Weekly , 2} + {V}_{Weekly , 3}$$

$${V}_{Total , accumulated} = 11 , 000 + 11 , 625 + 12 , 250$$

$${V}_{Total , accumulated} = 34 , 875 lb$$

Total Muscle Growth after 3 weeks:

$${G}_{total} = {G}_{1} + {G}_{2} + {G}_{3}$$

$${G}_{total} = 0.011 + 0.011625 + 0.0 \; 1225 {G}_{total} = 0.0 \; 34875 lb$$

Percentage of Increase in Muscle Mass after 3 weeks:

Percentage of increase__{Accumulated, 3}

$$Percentage of {increase}_{\_ Accumulated , 3} Percentage of {increase}_{\_ Accumulated , 3} = ( {G}_{\_ total} / {M}_{\_ Initial} ) \times 100 = ( 0.034875 / 127.5 ) \times 100 = 0.0274 \% .$$

Further, throughout three weeks of training with a weekly progression of 5 pounds, the user may see how the volume of training increases and how this translates into incremental muscle growth. The application of the FCMP allows the user to calculate the total accumulated volume and the percentage of increase in muscle mass in a systematic and quantifiable way. This approach provides a clear metric of progress and may be adjusted according to the individual needs and objectives of the athlete.

Further, adjusting the Progressive Muscle Growth Formula (FCMP) to reflect different levels of exercise intensity using the MET (Metabolic Equivalent of Task) value. This adjustment is applied using the initial muscle mass to calculate the percentage of increase in muscle mass.

Further, MET is a unit that estimates the amount of energy a person spends during a physical activity compared to being at rest. A MET of 8, for example, means that physical activity is using 8 times the energy it would use at rest. Formula for the Adjusted Factor (k)

Further, to adjust the value of (k) depending on the MET, the following formula is used:

$${K}_{\_ Adjusted} = {K}_{\_ base} \times MET$$

Where:
(K__{base}) Is the original proportionality factor (0.000001 in our example).
(MET) Is the value of the MET for the activity (in this case, 8).

Further, the value of (k) in the FCMP is a proportionality factor that converts the volume of training into muscle growth. Since the MET value reflects the intensity of the exercise, (k) may be adjusted to reflect this additional intensity.

Further, calculation of the Adjusted Factor (k)

$$Given a MET value of 8 : K\_Adjusted = 0.000001 \times 8 = 0.000008$$

Further, application of the adjusted FCMP:

### Initial Data—

For this example, the following parameters may be used:
Training frequency: 5 Days a week
Weekly progression: Increase of 5 pounds each week
Base proportionality factor (K_base): 0.000001
Adjusted proportionality factor (K_Adjusted): 0.000008
Initial muscle mass estimate (M_Initial): 127.5 lb
Valor MET (MET): 8

### Week 1

### Weights Used:

First set of approximation: 60 pounds×5 repetitions
Second series of approximation: 80 pounds×5 repetitions
Series with real weight: 100 pounds×5 repetitions×3 sets

### Calculation of the Daily Volume:

$${V}_{Daily , 1} = ( 60 lb \times 5 ) + ( 80 lb \times 5 ) + ( 100 lb \times 5 \times 3 )$$

$${V}_{Daily , 1} = 300 + 400 + 1 , 500$$

$${V}_{Daily , 1} = 2 , 200 lb$$

### Calculation of the Weekly Volume:

$${V}_{Weekly , 1} = {V}_{D i a r y , 1} \times 5 {V}_{W e e k l y , 3} = 2 , 200 lb \times 5 {V}_{W e e kly , 3} = 11 , 000 lb$$

Calculation of Adjusted Weekly Muscle Growth (G_₁):

$${G}_{1} = {K}_{\_ Adjusted} \times {V}_{Weekly , 1} G 1 = 0.000008 \times 11 , 000 {G}_{1} = 0.088 lb$$

Percentage of Increase in Muscle Mass after 1 week

To calculate the percentage of increase in muscle mass, the initial muscle mass (M__{Initial}) of 127.5 lb may be used:

$$Percentage of {increase}_{\_ Accumulated} , 1$$

$$( {G}_{\_ total} / {M}_{\_ Initial} ) \times 100 -> ( 0.088 / 127.5 ) \times 100 = ( 0.088 / 127.5 ) \times 100.$$

Further, by adjusting the factor (k) according to the MET value, the greater intensity of the exercise may be reflected in the calculation of muscle growth. This setting allows the FCMP to be more accurate for different levels of exercise intensity.

Further, the adjustment of factor k based on MET and using the initial muscle mass allows a more accurate estimate of muscle growth, considering both the volume of training and the intensity of the exercise.

Further, The Progressive Muscle Growth Formula (FCMP) offers a systematic and quantifiable framework for estimating muscle growth based on a progressive training routine. Further, the Components are as follows.

Daily Training Volume (V__{Diary}):

$${V}_{D iary , n} = ( {W}_{1 , n} \times {R}_{1} ) + ( {W}_{2 , n} \times {R}_{2} ) + ( {W}_{real , n} \times {R}_{real} \times {S}_{real} )$$

Evaluates the daily effort based on the weights and repetitions used.

Weekly Training Volume (V__{Weekly}):

$${V}_{\_ Weekly} = {V}_{\_ Diary} \times F$$

Expand the daily effort to a weekly perspective.

### Weekly Muscle Growth (G_ₙ):

$${G}_{\_ n} = K \times {V}_{\_ Weekly , n}$$

Percentage of Increase in Muscle Mass (Percentage of increase__{Accumulated, n}):

Translate the volume of training into muscle growth using a proportionality factor.

$$Total Accumulated Volume ( {V}_{\_ Total , accumulated} ) : {V}_{\_Total , accumulated} \text{\textasciicircum \textasciicircum} = {V}_{weekly , n}$$

Further, the weekly efforts are added up to provide a global view of training over time.

Further, calculating the impact of training in terms of the percentage of muscle mass gained through the examples. Further, throughout the examples provided, FCMP may be used to calculate the total accumulated volume and the percentage of increase in muscle mass. This approach allows athletes and coaches to monitor progress in a quantifiable way, adapting the training routine as necessary to achieve specific objectives.

Further, The FCMP provides a powerful tool for those who seek to maximize their muscle growth in a systematic and data-based way. By following this methodology, a quantifiable estimate of the progress in strength training may be obtained, adjusting the focus according to the individual needs and objectives of the athlete

Further, calculating and applying the formulas of ideal weight, calories burned at rest (CQR) and percentage of body fat (PGC). These formulas are essential tools for those who seek to improve their health and well-being through the management of their weight and the optimization of their body composition.

Further, Importance of Ideal Weight, CQR and Percentage of Body Fat are as follows. Further, the ideal weight is an estimate of the optimal body weight based on various factors such as height, sex, and age and body composition. Maintaining an ideal weight is crucial for several reasons. Further, General Health is a body weight within the ideal range is associated with a lower risk of chronic diseases such as type 2 diabetes, heart disease and certain types of cancer. Further, Physical Performance may include Reaching and maintaining the ideal weight may improve efficiency and performance in physical and sports activities. Further, Psychological Well-being is a healthy body weight contributes to better self-esteem and mental well-being. Further, Calories Burned at Rest (CQR) represent the amount of energy the body needs to maintain its vital functions while it is at rest. Calculating the CQR is important because knowing the CQR helps to establish an adequate daily caloric intake to maintain, gain or lose weight according to personal goals. Further, The CQR provides a basis for understanding basal metabolism and how different factors (such as muscle mass and age) affect it. Further, adjusting the caloric intake based on the COR may help avoid unwanted weight gain or loss. Further, the percentage of body fat (PGC) is a measure of the proportion of fat in the body compared to the total mass. Knowing the PGC is essential for several reasons. Further, The PGC offers a more accurate view of body composition than simply measuring total body weight. Further, A PGC that is too high or too low may be associated with an increased risk of diseases such as obesity, heart disease and hormonal problems. Further, the PGC helps establish and monitor specific fitness goals, such as fat loss or increased muscle mass.

Further, Ideal weight, calories burned at rest and body fat percentage are key metrics to understand and improve health and well-being.

Further, Ideal weight is an estimate of the optimal body weight for a person, based on several factors such as height, sex, age and body composition. It is not a single fixed number, but a healthy weight range that may vary between individuals. The ideal weight is calculated considering height sex, weight and body decomposition. Further, the relationship between height and weight is essential to determine the ideal weight. Further, Men and women have different body compositions and distributions of muscle mass and fat. Further, Body composition changes with age, affecting the ideal weight. Further, The proportion of muscle mass and fat in the body.

Calculating and maintaining the ideal weight is crucial for overall health, physical performance and psychological well-being. A weight within the ideal range reduces the risk of chronic diseases, improves efficiency in physical activities and contributes to better self-esteem.

Further, Calories burned at rest (CQR), also known as basal metabolic rate (BMT), represent the amount of energy the body needs to maintain basic vital functions while at rest. These functions include breathing, blood circulation, the functioning of organs and the regulation of body temperature.

Further, The CQR is important because of several reasons. Further, Knowing the CQR allows the user to establish an adequate caloric intake to maintain, gain or lose weight according to personal objectives. Further, CQR provides a basis for understanding basal metabolism and how different factors such as muscle mass, age and sex affect it. Further, adjusting caloric intake based on the CQR helps prevent unwanted weight gain or loss.

Further, the percentage of body fat (PGC) is a measure that indicates the proportion of fat in the body compared to the total mass. It is expressed as a percentage and is a key metric for evaluating body composition. The PGC is calculated taking into account. Further, Total Weight may include the total body weight of the person. Further, Fat Mass may include the total amount of fat in the body. Further, Lean Mass may include the body mass that is not fat, including muscles, bones, water and other tissues. Further, the PGC is essential for several reasons: Further, Evaluation of Body Composition offers a more accurate view of body composition than simply measuring total body weight. Further, there is Risk of Diseases if A PGC too high or too low.

Further, Muscle mass refers to the amount of muscle in the body. It is a crucial factor that affects the ideal weight, calories burned at rest (CQR) and the percentage of body fat. Muscle mass is more metabolically active than fat, which means that it consumes more energy to maintain itself. Further, in Ideal Weight, Greater muscle mass may increase the ideal weight due to its density and volume. Further, in CQR, People with greater muscle mass burn more calories at rest. Further, in Percentage of Body Fat, Greater muscle mass generally reduces the percentage of body fat.

Further, the somatotype is the classification of the human body according to physical structure and body composition. There are three main somatotypes: ectomorph, mesomorph and endomorphic. Further, Ectomorph may include thin people with less body fat and muscle mass. Further, Mesomorph may include People with a muscular and athletic structure, balance between muscle mass and body fat. Further, Endomorph may include People with a greater tendency to accumulate fat and a rounder body structure. Further, each somatotype influences how fat and muscle mass are distributed, affecting the ideal weight, the CQR and the percentage of body fat.

Further, Age significantly affects metabolism and body composition. As the user gets older, muscle mass tends to decrease and body fat tends to increase. Further, in Ideal Weight, it may vary with age due to changes in muscle mass and bone density. Further, in CQR, the basal metabolism decreases with age, reducing the calories burned at rest. Further, body fat percentage increases with age due to decreased muscle mass and hormonal changes.

Further, Lifestyle includes physical activity, diet and other daily habits that affect general health. Further, in Ideal Weight, an active lifestyle helps maintain a healthy muscle mass and an ideal weight. Further, in CQR, Regular physical activity increases the CQR. Further, in Body Fat Percentage, a healthy lifestyle helps maintain an adequate percentage of body fat.

Further, Stress affects metabolism and may influence body composition. Further, in Ideal Weight, Chronic stress may lead to unhealthy weight gain. Further, in CQR, Stress may alter metabolism and calorie burning. Further, in Percentage of Body Fat: Stress may contribute to the increase in body fat, especially in the abdominal region.

Further, Hydration is essential to maintain metabolism and body function. Further, in Ideal Weight: Dehydration may temporarily influence body weight. Further, in CQR, Good hydration is crucial for efficient metabolism and calorie burning. Further, in Body Fat Percentage, maintaining adequate hydration helps maintain a healthy body composition.

Further, Medical conditions may affect metabolism, weight and body composition. Further, in Ideal Weight, some medical conditions may require adjustments in the ideal weight. Further, in COR, Metabolic, hormonal and other medical conditions may affect the CQR. Further, in Body Fat Percentage, Medical conditions may influence the accumulation and distribution of body fat.

Further, the climate and the environment may influence metabolism and body composition. Further, in Ideal Weight, the climate may affect physical activity and, therefore, the ideal weight. Further, in CQR, People in cold climates may have a slightly higher CQR due to the energy needed to maintain body temperature. Further, in Percentage of Body Fat Climate may influence eating habits and the level of physical activity, affecting the percentage of body fat. Further, these factors play a crucial role in determining the ideal weight, the CQR and the percentage of body fat, and should be considered when evaluating a person's overall health and well-being.

Further, the formula for calculating ideal weight (PI) provides a personalized estimate of the optimal body weight to maintain good health and physical performance. The formula takes into account several individual factors, including muscle mass, somatotype, age, lifestyle, perceived stress level, hydration level and medical conditions.

### For Men:

$${PI}_{\_ h} = ( 50 + 0.9 \times ( Height ( cm ) - 150 ) ) \times Adjustments$$

### For Women:

$${PI}_{\_ m} = ( 45 + 0.8 \times ( Height ( cm ) - 150 ) ) \times Adjustments$$

Further, Muscle mass influences the ideal weight due to its higher density and energy requirement compared to body fat.

### For Men:

Low Muscle Mass: 1.0
Low Average Muscle Mass: 1.08
Medium High Muscle Mass: 1.10
High Muscle Mass: 1.12
Very High Muscle Mass: 1.13

### For Women:

Low Muscle Mass: 1.0
Average Low Muscle Mass: 1.07
Medium High Muscle Mass: 1.09
High Muscle Mass: 1.11
Very High Muscle Mass: 1.12

Further, the somatotype affects the distribution of fat and muscle mass in the body.
Ectomorph: 1.05
Mesomorph: 1.0
Endomorph: 0.95

Further, Age affects metabolism and body composition, influencing the ideal weight.

Child (0-12 years old): 0.8, Teenager (13-17 years old): 0.9, Adult (18-64 years old): 1.0, and Older Adult (65+ years old): 0.9

Further, Lifestyle, including physical activity, affects muscle mass and body fat.
Sedentary: 0.9
Moderately Active: 1.0
Very Active: 1.1

Further, Stress may influence metabolism and fat storage.
Low: 1.0, Moderate: 0.95, and High: 0.9

Further, Adequate hydration is essential for the optimal functioning of the body.
Suitable: 1.0
Inadequate: 0.95

Further, Medical conditions may affect metabolism and body composition.
No Medical Conditions: 1.0
With Medical Conditions: 0.9

Further, below are detailed examples of how to calculate the ideal weight for a man and a woman.

### Example for a Men:

### Data:

Age: 40 years' old
Height: 180 cm
Muscle Mass: High (1.12)
Somatotype: Mesomorph (1.0)
Lifestyle: Moderately Active (1.0)

### Calculation:

### Base for Ideal Weight (BPI):

$$BPI = 50 + 0.9 \times ( 180 - 150 ) BPI = 50 + 0.9 \times 30 BPI = 50 + 27 BPI = 77 kg$$

### Adjustments:

$$Adjustments = Muscle Mass ( 1.12 ) \times Somatotype ( 1. )$$

### Ideal Weight (PI):

$$PI = 77 \times Adjustments PI = 77 \times 1.12 PI = 86.24 kg$$

### Example for a Woman: Data:

Age: 35 years' old
Height: 165 cm
Muscle Mass: High (1.11)
Somatotype: Mesomorph (1.0)
Lifestyle: Moderately Active (1.0)
Perceived Stress Level: Moderate (0.95)
Hydration Level: Adequate (1.0)
Medical Conditions: No Medical Conditions (1.0)

### Calculation:

### Base for Ideal Weight (BPI):

$$BPI = 45 + 0.8 \times ( 165 - 150 ) BPI = 45 + 0.8 \times 15 BPI = 45 + 12 BPI = 57 kg$$

### Adjustments:

$$Adjustments = Muscle Mass ( 1.11 ) \times Somatotype ( 1. )$$

### Ideal Weight (PI):

$$PI = 57 \times Adjustments PI = 57 \times 1.11 PI = 63.27 kg$$

Further, calculating the ideal weight is a process that considers multiple individual factors. Using a formula that adjusts by muscle mass, somatotype, age, lifestyle, stress level, hydration and medical conditions allows the user to obtain a more accurate and personalized estimate of the ideal weight. This knowledge is essential to set clear objectives and design effective weight maintenance programs, thus improving general health and well-being.

Further, Calories burned at rest (CQR) represent the amount of energy that the body needs to maintain vital functions while it is in a state of rest. This calculation is crucial to understand the daily energy needs and adjust the caloric intake according to the health and fitness objectives. The general formula for calculating the CQR incorporates multiple adjustment factors to provide a personalized estimate.

### For Men:

$$CQR = ( Lean Body Mass ( MCM ) \times Adjustment Factors ) \times 25$$

### For Women:

$$CQR = ( Lean Body Mass ( MCM ) \times Adjustment Factors ) \times 23$$

Further, Incorporation of Current Weight and Percentage of Body Fat. Lean body mass (MCM) is calculated using the current weight and the percentage of body fat (PGC): MCM=Current Weight (kg)×(1−PGC+100)

Further, Adjustments for muscle mass are applied to reflect the amount of muscle in the body, since the muscle is more metabolically active than fat.

### Men:

Low Muscle Mass: 1.0
Low Average Muscle Mass: 1.08
Medium High Muscle Mass: 1.10
High Muscle Mass: 1.12
Very High Muscle Mass: 1.13

### Women:

Low Muscle Mass: 1.0
Average Low Muscle Mass: 1.07
Medium High Muscle Mass: 1.09
High Muscle Mass: 1.11
Very High Muscle Mass: 1.12

Further, the somatotype (body type) affects the distribution of muscle mass and body fat.
Ectomorph: 1.05
Mesomorph: 1.0
Endomorph: 0.95

Further, the metabolic rate decreases with age, so adjustments are applied to reflect this change.

Child (0-12 years old): 0.8, Teenager (13-17 years old): 0.9, Adult (18-64 years old): 1.0, and Older Adult (65+ years old): 0.9

Further, Lifestyle affects daily energy expenditure.
Sedentary: 0.9
Moderately Active: 1.0
Very Active: 1.1

Further, Stress may affect the metabolism.
Low: 1.0 Moderate: 0.95 High: 0.9

Further, Adequate hydration is crucial for metabolic functioning.
Suitable: 1.0 Inadequate: 0.95

Further, Medical conditions may influence basal metabolism.
No Medical Conditions: 1.0
With Medical Conditions: 0.9

Further, the climate affects the body's energy expenditure.
Temperate Weather: 1.0
Warm Weather: 1.1
Cold weather: 0.9

Further, below are detailed examples of how to calculate the calories burned at rest for a man and a woman, taking into account all the adjustment factors mentioned.

### Example for a Man:

### Data:

Current Weight (PA): 90 kg
Percentage of Body Fat (PGC): 20%
Muscle Mass: High (1.12)
Somatotype: Mesomorph (1.0)
Age: 40 years old (1.0)
Lifestyle: Moderately Active (1.0)
Perceived Stress Level: Moderate (0.95)
Hydration Level: Adequate (1.0)
Medical Conditions: No Medical Conditions (1.0)
Climate: Warm (1.1)

### Calculation:

### Lean Body Mass (MCM):

$$MCM = PA \times ( 1 - PGC - 100 ) MCM = 90 \times ( 1 - 20 - 100 ) MCM = 90 \times 0.8 MCM = 72 kg$$

### Adjustments:

$$Adjustments = 1.12 \times 1. \times 1. \times 1. \times 0.95 \times 1. \times 1. \times 1.1 Adjustments = 1.1716$$

### Calories Burned at Rest (CQR):

$$CQR = ( 72 \times Adjusts ) \times 25 CQR = ( 72 \times 1.1716 ) \times 25 CQR = 84.35 \times 25 CQR = 2 , 108.88 Calories per day$$

### Example for a Woman:

### Data:

Current Weight (PA): 70 kg
Percentage of Body Fat (PGC): 25%
Muscle Mass: High (1.11)
Somatotype: Mesomorph (1.0)
Age: 35 years old (1.0)
Lifestyle: Moderately Active (1.0)
Perceived Stress Level: Moderate (0.95)
Hydration Level: Adequate (1.0)
Medical Conditions: No Medical Conditions (1.0)
Climate: Warm (1.1)

### Further, Calculations:

### Lean Body Mass (MCM):

$$MCM = PA \times ( 1 - PGC - 100 ) MCM = 70 \times ( 1 - 25 - 100 ) MCM = 70 \times 0.75 MCM = 52.5 kg$$

### Adjustments:

$$Adjustments = 1 \text{.11} \times 1. \times 1. \times 1. \times 0.95 \times 1. \times 1. \times 1.1 Adjustments = 1.16055$$

$$CQR = ( 52.5 \times Adjusts ) \times 23 CQR = ( 52.5 \times 1.16055 ) \times 23 CQR = 60.93 \times 23 CQR = 1401.55 Calories per day$$

Further, calculating the calories burned at rest is a process that considers multiple individual factors, including the percentage of body fat and the current weight. Using a formula that adjusts by muscle mass, somatotype, age, lifestyle, stress level, hydration, medical conditions and weather allows the user to obtain a more accurate and personalized estimate of daily caloric needs at rest. Knowing this value is essential to properly plan diet and exercise, thus promoting better general health and well-being.

Further, Daily water intake (IDA) is the amount of water that a person must consume daily to stay hydrated and support essential body functions. The formula for calculating the IDA considers several factors such as muscle mass, somatotype, age, lifestyle, perceived stress level, hydration level, medical conditions and climate.

Further, General Formula to Calculate the IDA:

$$IDA = ( Current Weight ( kg ) \times Adjustments ) \times 0.035$$

$$IDA = ( Current Weight ( kg ) \times Adjustment by Muscle Mass \times Adjustment by Somatotype \times Adjustment by Age \times Adjustment by Lifestyle \times Adjustment by Stress \times Adjustment by Hydration \times Adjustment by Medical Conditions \times Adjustment by Climate ) \times Hydration Factor$$

Conversion from ml to L:

$$1 liter ( L ) = 1000 milliliters ( ml )$$

$$35 ml = 35 ml / 1000 = 0.0 \; 35 L$$

Therefore, multiplying by 0.035 represents 35 ml per kg of body weight.

Muscle mass affects the amount of water needed due to its higher metabolic demand compared to body fat.

Low Muscle Mass: 1.0, Low Average Muscle Mass: 1.08, Medium High Muscle Mass: 1.10, High Muscle Mass: 1.12, and Very High Muscle Mass: 1.13.

Further, the somatotype (body type) influences water needs due to differences in body composition.
Ectomorph: 1.05
Mesomorph: 1.0
Endomorph: 0.95

Further, Water needs vary according to age due to changes in body composition and metabolism.

Child (0-12 years old): 0.8 Teenager (13-17 years old): 0.9 Adult (18-64 years old): 1.0 Older Adult (65+ years old): 0.9

Further, the level of physical activity affects the amount of water needed to maintain hydration.
Sedentary: 0.9
Moderately Active: 1.0
Very Active: 1.1

Further, Stress may influence water needs due to its effects on metabolism and fluid loss.
Low: 1.0 Moderate: 0.95 and High: 0.9

Further, the current hydration level affects the amount of water needed to maintain homeostasis.

Suitable: 1.0 Inadequate: 0.95 Further, Medical conditions may alter water needs due to changes in physiology and metabolism.
No Medical Conditions: 1.0
With Medical Conditions: 0.9

Further, the climate affects the amount of water that is lost through perspiration and, therefore, the water needs.
Temperate Weather: 1.0, Warm Weather: 1.1, and Cold weather: 0.9
Further, Examples of IDA Calculation

### Example for a Man:

### Data:

Current Weight (PA): 90 kg
Muscle Mass: High (1.12)
Somatotype: Mesomorph (1.0)
Age: 40 years old (1.0)
Lifestyle: Moderately Active (1.0)
Perceived Stress Level: Moderate (0.95)
Hydration Level: Adequate (1.0)
Medical Conditions: No Medical Conditions (1.0)
Climate: Warm (1.1)

### Further, Calculations:

$$Adjustments = 1.12 \times 1. \times 1. \times 1. \times 0.95 \times 1. \times 1. \times 1.1 Adjustments = 1.1716$$

$$IDA \text{:} IDA = ( 90 \times 1.1716 ) \times 0.035 IDA = 105.444 \times 0.035 IDA = 3.69 Liters per day$$

Example for a woman: Data:
Current Weight (PA): 70 kg
Muscle Mass: Medium High (1.09)
Somatotype: Mesomorph (1.0)
Age: 35 years old (1.0)
Lifestyle: Moderately Active (1.0)
Perceived Stress Level: Moderate (0.95)
Hydration Level: Adequate (1.0)
Medical Conditions: No Medical Conditions (1.0)
Climate: Warm (1.1)

### Further, Calculation:

$$Adjustments = 1.09 \times 1. \times 1. \times 1. \times 0.95 \times 1. \times 1. \times 1.1 Adjustments = 1.13895$$

### IDA:

$$IDA = ( 70 \times 1.13895 ) \times 0.035 IDA = 79.7265 \times 0.035 IDA = 2.79 Liters per day$$

Further, calculating daily water intake (DIDA) is a process that considers multiple individual factors. Using a formula that adjusts by muscle mass, somatotype, age, lifestyle, stress level, hydration, medical conditions and weather allows the user to obtain a more accurate and personalized estimate of daily hydration needs. Knowing this value is essential to maintain good health and optimal performance.

Further, the percentage of excess fat (PGE) is a measure that helps identify the amount of additional body fat that a person has compared to their ideal weight. Calculating the PGE is crucial to plan weight loss programs and improve overall health, since excess body fat may increase the risk of various diseases.

Further, the formula for calculating the percentage of excess fat is based on comparing the current weight with the ideal weight. This formula provides an estimate of the percentage of the current body weight that is made up of excess fat.

$$PGE = ( Current Weight ( kg ) - Ideal Weight ( kg ) ) - Current Weight ( kg ) \times 100.$$

Further, Importance of PGE in Health and the Planning of Weight Loss Programs. Further, knowing the percentage of excess fat has several advantages, including Body Health Assessment. Further, it allows the user to evaluate the body composition more precisely instead of relying only on the total weight. Further, it helps identify possible health risks associated with excess body fat, such as cardiovascular diseases, type 2 diabetes and certain types of cancer. Further, Planning of Weight Loss Programs helps to set clear and measurable goals for the loss of body fat. Further, it provides a basis for designing personalized and effective weight loss programs. Further, it facilitates the evaluation of progress in the loss of body fat and allows necessary adjustments in the diet and exercise regime. Further, it facilitates the monitoring of progress in weight loss and fitness programs. Further, it allows the user to evaluate the effectiveness of health interventions and adjust the plans as necessary.

Further, below are detailed examples of how to calculate the percentage of excess fat for a man and a woman.

### Example for a Men:

### Data:

Current Weight (PA): 90 kg
Ideal Weight (PI): 80 kg

### Calculation:

### Percentage of Excess Fat (PGE):

$$PGE = ( 90 - 80 ) - 90 \times 100 PGE = ( 10 - 90 ) \times 100 PGE = 0.111 \times 100 PGE = 11.11 \%$$

Further, the interpretation is that the man has 11.11% excess body fat. This value indicates the amount of additional fat the user should lose to reach the users ideal weight.

### Example for a Woman:

### Data:

Current Weight (PA): 70 kg
Ideal Weight (PI): 60 kg

$$PGE = ( 70 - 60 ) - 70 \times 100 PGE = ( 10 - 70 ) \times 100 PGE = 0.1429 \times 100 PGE = 14.29 \%$$

Further, the interpretation is that the woman has 14.29% excess body fat. This value indicates the amount of additional fat the user should lose to reach the ideal weight.

Further, calculating the percentage of excess fat is an important step to understand body composition and properly plan fat loss. Using a formula that compares the current weight with the ideal weight provides an accurate estimate of the amount of additional body fat. This knowledge is essential to set clear goals and design effective fat loss programs, thus improving general health and well-being.

Further, the percentage of ideal body fat (PGI) is a measure that indicates the optimal amount of body fat that a person must have to maintain good health and optimal physical performance. This value varies according to factors such as sex, age, and muscle mass and exercise frequency. To calculate the PGI, the system may use a formula that incorporates these factors to provide an accurate and personalized estimate.

Further, the General Formula is as follows:
Adjusted PGI=PGI Base×(Exercise Frequency Factor−Muscle Mass Factor)

### Adjustment Factors for Muscle Mass Men:

Low Muscle Mass: 1.0
Low Average Muscle Mass: 1.08
Medium High Muscle Mass: 1.10
High Muscle Mass: 1.12
Very High Muscle Mass: 1.13

### Women:

Low Muscle Mass: 1.0
Low Average Muscle Mass: 1.07
Medium High Muscle Mass: 1.09
High Muscle Mass: 1.11
Very High Muscle Mass: 1.12

### Adjustment Factors for Exercise Frequency Men:

Sedentary: 0.99
1-2 Times a week: 0.85
3-4 Times a week: 0.80
5-6 Times a week: 0.69
7 Times a week: 0.60

### Women:

Sedentary: 0.95
1-2 Times a week: 0.75
3-4 Times a week: 0.71
5-6 Times a week: 0.69
7 Times a week: 0.67

Further, importance of PGI in Health and the Planning of Physical Conditioning Programs. Further, knowing the ideal fat percentage is crucial for several reasons. Further, Body Health Assessment allows the user to evaluate the body composition more precisely instead of relying only on the total weight. Further, it helps to identify possible health risks associated with excess or deficit of body fat. Further, Planning of Physical Conditioning Programs helps to set clear and measurable goals for the loss or gain of body fat. Further, it facilitates the personalization of exercise and nutrition programs to achieve and maintain the PGI. Further, Monitoring of Progress facilitates the monitoring of progress in fitness and weight loss programs. Further, it helps to make the necessary adjustments to maintain health and optimal physical performance.

Further, below is a detailed example of how to calculate the ideal adjusted fat percentage for a man and a woman.

Example for a man who Exercises 6-7 Times a Week and Has High Muscle Mass:

### Data:

Age: 40 years' old
Gender: Male
Ideal Weight (PI): 80 kg
Activity Level: Very Active (6-7 times per week)
Muscle Mass: High (1.12)
Exercise Frequency: 7 times per week (0.60)

### Calculation:

### Percentage of Ideal Fat (PGI Base):

According to the table, for a very active 40-year-old man, the ideal range is 11-21%.

We may use the average of the range to obtain a representative value:

$$PGI Base = ( 11 + 21 ) - 2 = 16 \%$$

### Adjustment by Muscle Mass and Exercise Frequency:

$$Adjusted PGI = 16 \% \times ( 0.6 - 1.12 ) Adjusted PGI = 16 \% \times 0.535 Adjusted PGI = 8.57 \%$$

### Ideal Body Fat (GCI) in Kilograms:

$$GCI = 80 \times ( 8.57 - 1. ) GCI = 80 \times 0.0857 GCI = 6.856 kg$$

Example for a woman who doesn't exercise and has low muscle mass: Data:
Age: 30 years' old

Sex: Female
Ideal Weight (PI): 60 kg
Activity Level: Sedentary
Muscle Mass: Low (1.0)
Frequency of Exercise: Sedentary (0.95)

### Calculation:

### Percentage of Ideal Fat (PGI Base):

According to the table, for a 30-year-old sedentary woman, the ideal range is 23-28%.

We may use the average of the range to obtain a representative value:

$$PGI Base = ( 23 + 28 ) - 2 = 25.5 \%$$

### Adjustment by Muscle Mass and Exercise Frequency:

$$Adjusted PGI = 25 \text{.5} \% \times ( 0.95 - 1. ) Adjusted PGI = 24.22 \%$$

### Ideal Body Fat (GCI) in Kilograms:

GCI=60×(24.22−100) GCI=60×0.225 GCI=14.53 further, calculating the percentage of ideal body fat adjusted by muscle mass and exercise frequency allows a more accurate and personalized estimate. This knowledge is essential to set clear objectives and design effective fitness and nutrition programs, thus improving health and general well-being.

Further, to calculate the necessary macronutrients, first determine the necessary daily calories (CDN). Then, the percentages of macronutrients (carbohydrates, proteins and fats) may be adjusted according to the desired goal: lose weight, gain weight, maintain, increase muscle mass or increase muscle mass and lose weight. Finally, the system may divide the daily intake of macronutrients by the number of preferred meals (3, 4 or 5).

Further, the general Formula is CDN=CQR×Activity Level, where CQR: Calories burned at rest. Activity Level: Factor based on physical activity (Sedentary: 1.2, Moderately Active: 1,375, Active: 1.55, Very Active: 1,725, Extremely Active: 1.9).

### Further, Distribution of Macronutrients:

### Losing Weight:

Carbohydrates: 40%
Proteins: 40%
Fats: 20%

### Gaining Weight:

Carbohydrates: 50%
Proteins: 25%
Fats: 25%

### Maintaining Weight:

Carbohydrates: 50%
Proteins: 30%
Fats: 20%

### Increase Muscle Mass:

Carbohydrates: 40%
Proteins: 35%
Fats: 25%

Further, Increase Muscle Mass and Lose Weight:

### Maintaining Weight:

Carbohydrates: 50%
Proteins: 30%
Fats: 20%

### Increase Muscle Mass:

Carbohydrates: 40%
Proteins: 35%
Fats: 25%

### Further, Increase Muscle Mass and Lose Weight:

Carbohydrates: 30%
Proteins: 50%
Fats: 20%

Further, formulas for Calculating Macronutrients

To calculate the grams of each macronutrient needed per day, use the following formulas:

$$Carbohydrates ( g ) = ( CDN percentage of Carbohydrates - 4 )$$

$$Proteins ( g ) = ( CDN \times Percentage of Proteins - 4 )$$

$$Fats ( g ) = ( CDN \times Percentage of Fats - 9 )$$

Further, to distribute macronutrients in 3, 4 or 5 meals a day:

$$Carbohydrates per meal ( g ) = Carbohydrates ( g ) - Number of meals$$

$$Protein per meal ( g ) = Protein ( g ) - Number of meals$$

$$Fats per meal ( g ) = Fats ( g ) - Number of meals$$

### Example 1: Man Who Wants to Increase Muscle Mass Data

Daily Calories Needed (CDN): 3000 cal
Objective: To Increase Muscle Mass
Number of Meals: 4
Distribution of Macronutrients:
Carbohydrates: 40%
Proteins: 35%
Fats: 25

Further, the calculation are as follows. Carbohydrates:

$$\begin{matrix}Carbohydrates ( g ) = 3 , 000 \times 0.4 - 4 \\ = 300 g \\ Carbohydrates per meal ( g ) = 300 - 4 \\ = 75 g\end{matrix}$$

### Proteins:

$$\begin{matrix}Proteins ( g ) = 3 , 000 \times 0.35 - 4 \\ = 262.5 g \\ Proteins for food ( g ) = 262.5 - 4 \\ = 65.62 g\end{matrix}$$

### Fats:

$$\begin{matrix}Fats ( g ) = 3 , 000 \times 0.25 - 9 \\ = 83.3 g \\ Fats for food ( g ) = 83.3 - 4 \\ = 20.82 g\end{matrix}$$

### Example 2: Woman Who Wants to Lose Weight

### Data:

Daily Calories Needed (CDN): 2000 cal
Objective: Lose Weight
Number of Meals: 3

### Distribution of Macronutrients:

Carbohydrates: 40%
Proteins: 40%
Fats: 20%

### Calculation: 1. Carbohydrates:

$$\begin{matrix}Carbohydrates ( g ) = 2 , 000 \times 0.4 - 4 \\ = 200 g \\ Carbohydrates per meal ( g ) = 200 - 3 \\ = 66.67 g\end{matrix}$$

$$2. Proteins :$$

$$\begin{matrix}Proteins ( g ) = 2 , 000 \times 0.4 - 4 \\ = 200 g \\ Proteins for food ( g ) = 200 - 3 \\ = 66.67 g\end{matrix}$$

### 3. Fats:

$$\begin{matrix}Fats ( g ) = 2 , 000 \times 0.2 - 9 \\ = 44.4 g \\ Fats for food ( g ) = 44.4 - 3 \\ = 14.8 g\end{matrix}$$

Further, determining the necessary macronutrients based on daily calories and the personal goal is essential to achieve health and physical performance goals. Using a formula that adjusts the distribution of macronutrients according to the desired objective and dividing them into the number of preferred meals, the system may customize the diet to optimize the results.

Further, when it comes to adjusting the daily calories needed (CDN) to achieve a specific health or fitness goal, such as losing weight, maintaining weight, gaining weight, gaining muscle mass or combining muscle mass gain with weight loss, it is essential to apply the correct adjustments to the daily calories needed (CDN).

Further, to lose weight, it is necessary to create a caloric deficit, which means consuming fewer calories than the body burns daily. A typical recommended deficit is 10-20%, which is safe and effective for most people.

$$Further , Adjusted Calories = CDN \times ( 1 - 20 - 100 )$$

Further, the formula reduces the daily calories needed (CDN) by 20%, which helps ensure that the body uses its fat reserves as an energy source to compensate for the caloric deficit.

Further, to maintain the current weight, the user must consume as many calories as the body burns daily. This implies maintaining a caloric balance without the need for additional adjustments.

$$Further , Adjusted Calories = C D N$$

Further, Here, no adjustment is made to the daily calories needed (CDN), since the goal is simply to maintain the current weight unchanged.

Further, to gain weight, the user need a caloric surplus, which means consuming more calories than the body burns daily. A typical recommended surplus is 10-20%, which facilitates a gradual and healthy weight gain.

$$Further , Adjusted Calories = CDN \times ( 1 + 20 - 100 )$$

Further, the formula increases the daily calories needed (CDN) by 20%, which provides the body with the excess energy needed to store in the form of mass (either fat or muscle, depending on the type of physical activity).

Further, to gain muscle mass, a slight caloric surplus is required, accompanied by an increase in protein intake. A typical recommended increase is 10-15%.

$$Further , Adjusted Calories = CDN \times ( 1 + 15 - 100 )$$

Further, the formula increases the daily calories needed (CDN) by 15%, providing the additional energy necessary for muscle growth, in combination with adequate strength training.

Further, this goal is challenging because it requires a precise approach, with a small caloric deficit (5-10%) while ensuring an adequate intake of proteins to preserve muscle mass.

$$Further , Adjusted Calories = CDN \times ( 1 - 10 - 100 )$$

Further, the formula reduces the daily calories needed (CDN) by 10%, which allows the loss of fat while minimizing the loss of muscle mass, as long as it is accompanied by adequate protein consumption and strength training.

Further, regardless of the objective, the distribution of macronutrients is crucial to ensure that the body obtains the necessary energy from the right sources. Below is a recommended standard distribution of macronutrients. Further, Carbohydrates is 50%. Further, Proteins is 20%. Further, Fats is 30%. Further, these percentages represent the proportion of the total calories that must come from each macronutrient. By dividing these percentages by 100, get the percentage factors that apply to the adjusted calories.

Further, formulas to Calculate Macronutrients is as follows. Further, using the calories adjusted according to the objective, the daily grams of each macronutrient may be calculated.

### Grams of Carbohydrates Formula:

$$Grams of Carbohydrates = ( Adjusted Calories \times 50 - 100 ) - 4 Explanation :$$

50% of the adjusted calories go to carbohydrates, and since each gram of carbohydrates provides 4 calories, the system may divide the total of carbohydrate calories by 4 to get the daily grams.

### Further, Grams of Proteins Formula:

$$Grams of Proteins = ( Adjusted Calories \times 20 - 100 ) - 4 Explanation :$$

20% of the adjusted calories are destined for protein, and since each gram of protein provides 4 calories, the system may divide the total of protein calories by 4 to obtain the grams per day.

### Grams of Fats Formula:

$$Grams of Fats = ( Adjusted Calories \times 30 - 100 ) - 9 ;$$

Explanation: 30% of the adjusted calories are allocated to fat, and since each gram of fat provides 9 calories, the system may divide the total fat calories by 9 to obtain the daily grams.

Further, understanding and applying these adjustments is crucial to achieving goals such as weight loss, muscle mass gain, or weight maintenance in an effective and healthy way.

Further, the general formula that integrates the percentage of body fat, the calories burned at rest (CQR), and the daily calories needed (CDN) to calculate the time needed to gain or lose a specific amount of weight is as follows:

### Calculation of Calories Burned at Rest Adjusted (Adjusted CQR)

Further, this part of the formula adjusts the calories burned at rest based on the percentage of body fat, taking into account that only lean mass is metabolically active.

$$Adjusted CQR = CQR \times ( 1 - PGC - 100 ) Where :$$

CQR: Calories burned at rest
PGC: Percentage of body fat

### Calculation of the Adjusted Daily Caloric Surplus or Deficit

The daily caloric surplus or deficit is obtained by subtracting the calories burned at rest adjusted from the necessary daily calories (CDN).

$$Survit or Daily Caloric Deficit Adjusted = CDN - CQR adjusted where :$$

CDN: Daily calories needed.

### Calculation of Pounds Earned or Lost per Day:

To convert the adjusted daily caloric surplus or deficit into pounds gained or lost per day, the following formula is used:

### Formula:

$$Pounds per Day = Adjusted Daily Caloric Surplus or Deficit - 3 , 500 Where :$$

3,500 calories are equivalent to approximately one pound of body weight.

Further, Calculation of the time required to gain or lose weight

Further, to determine the time required to gain or lose a specific amount of weight, the formula is used: Time (days)=Pounds to Gain or Lose-Pounds Per Day.

Further, Pounds to Earn or Lose may include amount of weight the user want to gain or lose. Complete General Formula. Further, by combining all these steps into a single formula, Time (days)=Pounds to Gain or Lose−(CDN−(CQR×(1−PGC−100))).

Further, COR is the number of calories that the body burns at rest, that is, the calories necessary to maintain the basic functions of the body (breathing, blood circulation, etc.).

Further, PGC is the percentage of body fat. This value is used to adjust the CQR, considering that only the lean mass of the body (muscles, organs, etc.) is metabolically active.

Further, CDN is the number of calories that the person consumes daily.

Further, 3500 is the amount of calories that is approximately equivalent to a pound of body weight.

Further, applying a general formula for a specific example to calculate the time it takes for a man of 190.5 cm in height, with a current weight of 210 pounds and a percentage of body fat of 10.9%, to gain 2.5 pounds. The additional data is that it burns 2679.7 calories at rest (CQR) and needs to consume 4776.6 calories daily (CDN) to achieve its goal.

Further, Data from the example may include Height: 190.5 cm.

Further, Current Weight: 210 pounds.

Further, Percentage of Body Fat (PGC): 10.9%.

Further, Pounds to Increase: 2.5 Pounds.

Further, Calories Burned at Rest (CQR): 2679.7 Calories.

Further, Daily Calories Needed (CDN): 4776.6 Calories.

Further, Step 1: Calculate the Calories Burned at Rest Adjusted (Adjusted QCR) using the formula:

CQR Adjusted=CQR×(1−PGC−100); Replacing the values:

$$Adjusted CQR = 2 , 679.7 \times ( 1 - 10.9 - 100 ) Adjusted CQR = 2 , 679.7 \times 0.891 = 2 , 388.25 carlories .$$

Further, Step 2: Calculate the Adjusted Daily Caloric Surplus.

### Replacing the Values:

$$Adjusted Daily Caloric Surplus = 477 6.6 - 238 \; 8.25 = 2388.35 calories .$$

Further, Step 3: Calculate the Pounds Earned per Day using the formula:

$$Pounds per Day = Adjusted Daily Caloric Surplus - 3 , 500 Replacing the values :$$

$$Pounds per day = 2388.35 calories - 3 , 500 calories = 0.682 Pounds / Day . Further ,$$

Step 4: Calculate the Time Needed to Gain 2.5 Pounds using the formula:

$$Time ( days ) = Pounds to Gain - Pounds per Day .$$

$$Further , Replacing the values : Time ( days ) = 2.5 Pounds - 0.682 Pounds = 3.67 Days .$$

Further, for this 210-pound man, with a 10.9% body fat percentage, to gain 2.5 pounds, it may take approximately 3.67 days (about 3 days and 16 hours) if he maintains an adjusted daily caloric surplus of 2388.35 calories.

Further, this example shows how the general formula may be applied to estimate the time needed to reach a specific weight goal, taking into account the body composition of the individual.

Further, below, it explains how to use these formulas in daily life, recommendations are offered to maintain an ideal weight and a percentage of healthy fat, the importance of continuous monitoring and adjustment is emphasized, and tips for adequate hydration are provided.

Further, to Use These Formulas in Daily Life, set your Goals: Further, determine if the user wants to lose weight, gain weight, maintain your current weight, and increase muscle mass, or a combination of these goals. Further, Use the Daily Calories Required (CDN) formula to calculate your daily caloric intake based on your goals and level of physical activity. Further, calculate your Macronutrients: Further, Apply the distribution of macronutrients (carbohydrates, proteins and fats) according to your goals. Further, divide the daily intake of macronutrients by the number of meals the user plan to eat (3, 4 or 5 meals a day). Further, Plan your meals. Further, Use the calculated amounts of macronutrients to plan your daily meals. Further, choose foods that fit your nutritional needs and personal preferences, making sure to include a variety of nutrient-rich foods. Further, Adapt the Formula to your Needs. Further, if the user notices that your energy needs or your body composition change, adjust the CDN formula and the distribution of macronutrients to reflect these changes.

Further, recommendations to maintain the ideal weight and a percentage of healthy fat may include eating in a balanced way. Further, include a variety of nutrient-rich foods in your daily diet, ensuring that the user meets all macronutrient and micronutrient needs. Further, prioritize the consumption of complex carbohydrates, lean proteins and healthy fats. Further, control the portions. Further, keep a control of the portions to avoid excessive calorie consumption, which could lead to unwanted weight gain. Further, use smaller dishes and serve yourself portions appropriate to your caloric needs. Further, incorporate physical activity. Further, exercise regularly, combining strength training with cardiovascular exercises to maintain a healthy percentage of fat and promote muscle mass. Further, adjust your caloric and macronutrient intake according to your level of physical activity. Further, avoid extreme diets. Further, opt for a balanced and sustainable approach instead of extreme diets that promise quick results but are difficult to maintain in the long term.

Further, importance of continuous monitoring and adjustment may include monitoring your progress: Further, keep a record of your weight, body fat percentage and other key health indicators to assess your progress. Further, use tools such as food tracking and physical activity applications to keep a detailed record. Further, make adjustments as needed: Further, if the user notices that your progress is stagnating or that the user is experiencing unwanted effects, adjust your caloric intake and distribution of macronutrients. Further, consult a health professional or a nutritionist if the user need help making these adjustments. Further, Be Patient and Persistent: Further, changes in body composition and weight may take time. Maintain consistency and don't be discouraged if the results are not immediate. Further, re-evaluate your goals regularly and adjust your plan as necessary to continue advancing towards your goals.

Further, tips for proper hydration may include consuming enough water: Further, drink at least 8 glasses of water a day, and adjust this amount according to your level of physical activity, the weather, and your personal needs. Further, be sure to drink water before, during and after exercise to stay hydrated. Further, incorporate liquids in your meals. further, complement your fluid intake with foods rich in water, such as fruits and vegetables. Further, consider consuming unsweetened drinks, such as herbal tea, to vary your fluid intake. Further, monitor the signs of dehydration. Further, pay attention to signs such as thirst, dark-coloured urine, and a feeling of fatigue, which may indicate dehydration. Further, increase your water intake if the user notices any of these symptoms, especially in warm climates or during intense exercise. Further, avoid excess sugary and alcoholic beverages further, limit the consumption of sugary and alcoholic beverages, which may dehydrate the user and add empty calories to your diet. Further, opt for healthier alternatives, such as water with fruit infusions or mineral water.

Further, the practical application of the formulas presented may help the user achieve and maintain health and fitness goals. By following the recommendations to maintain the ideal weight, adjust your intake as needed, and maintain adequate hydration, the user may be on your way to improving your general well-being and leading a healthy lifestyle.

Further, the formulas and strategies presented offer a comprehensive approach to achieving and maintaining your health and fitness goals. By using these formulas to calculate the daily calories needed, adjust the intake of macronutrients according to your goals, and divide this intake into practical meals, you may design a personalized and sustainable nutritional plan. The key to success lies in the constant and adaptive application of these principles to daily life.

Further, defining whether the user want to lose weight, gain weight, maintain weight, increase muscle mass or a combination of these goals is the first step to developing an effective plan.

Further, using the formulas to calculate the daily calories needed and adjust the macronutrients allows the user to make sure that the user is getting the energy and nutrients the user need.

Further, Meal Planning may include dividing the daily intake of macronutrients into 3, 4 or 5 meals may facilitate the fulfilment of your goals and maintain adherence to your nutritional plan. Further, Continuous monitoring of the user progress and the willingness to adjust intake as needed are essential to maintain the course towards the goals. Further, staying properly hydrated is essential for general health and physical performance.

Further, balanced and sustainable approach may include opting for a balanced and sustainable diet, instead of quick and extreme solutions, guarantees long-term results and improves general well-being.

Further, nutrition and exercise are only parts of a healthy lifestyle. Proper rest, stress management and mental well-being also play crucial roles in your overall health. Adopting a holistic and balanced approach to your well-being may help the user achieve and maintain your health goals effectively.

Further, consultation with professionals is always advisable to consult with health professionals, such as nutritionists and doctors, especially if the user have pre-existing medical conditions or special dietary needs. They may offer the user personalized guidance and make sure that your nutrition plan is safe and effective.

Further, by applying the principles and formulas, the user is taking an important step towards better health and physical condition. Consistency, patience and willingness to adapt over time may allow the user to achieve your goals and enjoy a healthier and more active life.

Further, Ideal Weight (PI) is an optimal body weight range determined based on several personal factors such as height, muscle mass, age, sex, and level of physical activity. It is the weight that is associated with a lower risk of chronic diseases and better physical performance.

Further, Calories Burned at Rest (CQR) represent the amount of energy that your body needs to maintain basic vital functions (breathing, circulation, temperature regulation, etc.) while the user is at rest. It is also known as the basal metabolic rate (BMD).

Further, Daily Water Intake (IDA) is the amount of water that a person must consume daily to stay hydrated and support essential body functions. This value is adjusted according to body weight, muscle mass, climate, and other factors.

Further, the Percentage of Excess Fat (PGE) is a measure that compares the amount of current body fat with what would be ideal for a person, based on their ideal weight. Indicates the amount of additional fat that a person must lose to reach a healthy range.

Further, the Ideal Fat Percentage (PGI) is the optimal percentage of body fat to maintain good health and physical performance. This percentage varies according to sex, age, muscle mass, and the level of physical activity.

Further, Lean Body Mass (MCM) is the weight of everything that makes up the body except fat. It includes muscles, bones, organs, and water. It is a key factor in the determination of calories burned at rest and in the planning of caloric intake.

Further, the somatotype is a classification that describes the structure and physical composition of a person. There are three main types. Further, Ectomorph may include people with a thin body and less body fat and muscle mass. Further, Mesomorph may include people with a muscular and athletic structure, balanced between muscle mass and body fat. Further, Endomorph may include people with a greater tendency to accumulate fat and a rounder body structure.

Further, basal metabolism is the amount of energy your body needs to perform vital functions while at rest. It is similar to Calories Burned at Rest (CQR) and is a key indicator of daily energy expenditure.

Further, factor adjustments are multipliers used in the formulas to customize the calculations according to individual characteristics such as muscle mass, somatotype, age, level of physical activity, stress, hydration, and medical conditions.

Further, macronutrients are the three main nutrients that the body needs in large quantities to function properly: carbohydrates, proteins, and fats. Each of these macronutrients has a specific function in the body and provides a different amount of calories per gram.

Further, the level of physical activity is a factor that describes how much exercise or activity a person performs in their daily life. It may be sedentary, moderately active, active, very active, or extremely active. This factor is crucial for calculating the daily calories needed (CDN).

Further, climate refers to the environmental conditions where a person lives, such as temperate, warm, or cold. The climate affects the amount of energy that the body needs to maintain its internal temperature, which in turn influences the amount of water and calories that are needed.

Further, perceived stress is the amount of stress that a person feels or experiences in their daily life. Stress may influence metabolism and the distribution of body fat, thus affecting calorie and hydration needs.

Further, medical conditions are health problems that can affect metabolism, body mass, and fat distribution, such as metabolic, hormonal, or chronic diseases. These conditions require specific adjustments in the calculations of the formulas.

Further, body composition refers to the proportion of fat mass and lean mass (muscle, bones, water) in the body. It is a more accurate indicator of physical health than body weight alone.

Further, formula to calculate the Ideal Weight (PI)

### For Men:

$${PI}_{\_ h} = ( 50 + 0.9 \times ( Height ( cm ) - 150 ) ) \times Adjustments$$

### For Women:

$${PI}_{\_ m} = ( 45 + 0.8 \times ( Height ( cm ) - 150 ) ) \times Adjustments$$

### Explanation of the Numbers:

50 (men) and 45 (women): These values are used as a basis to calculate the ideal weight in people 150 cm tall. The difference between men and women reflects the average variations in body composition, with men typically having more muscle mass and higher bone density.
0.9 And 0.8: These coefficients represent the increase in the ideal weight for each centimetre of height over 150 cm. The values are slightly different for men and women to reflect the typical differences in body composition.
150 cm: This is the height reference point used to start the calculation. Adjustments are applied from this height to calculate the ideal weight for taller or shorter people.
Adjustments: Adjustment factors are applied to customize the ideal weight according to individual characteristics such as muscle mass, somatotype, age, lifestyle, among others.

Further, formula for calculating Calories Burned at Rest (CQR):

### For Men:

$$CQR = ( Lean Body Mass ( MCM ) \times Adjustment Factors ) \times 25$$

### For Women:

$$CQR = ( Lean Body Mass ( MCM ) \times Adjustment Factors ) \times 23$$

### Explanation of the Numbers:

25 (men) and 23 (women): These values are based on the average basal metabolic rate, which estimates how many calories per kilogram of lean body mass (CMM) are burned per day. The difference between men and women reflects the variations in the metabolic rate due to differences in body composition and size.
MCM (Lean Body Mass): Represents the total mass of the body minus body fat. This value is crucial because lean mass (muscles, organs, bones) burns more calories than fat mass, even at rest.
Adjustment Factors: These factors allow the user to adjust the CQR according to individual variables such as muscle mass, somatotype, age, lifestyle, etc.

Formula to calculate Lean Body Mass (MCM):

$$MCM = Current Weight ( kg ) \times ( 1 - Percentage of Fat + 100 )$$

### Explanation of the Numbers:

Current Weight (kg): This is the total body weight of the individual.
(1−Percentage of Fat+100): This calculation converts the percentage of body fat into a decimal factor (for example, 20% is converted to 0.20) and subtracts it from 1 to determine the fraction of the total weight that is lean mass (muscle, bone, organs).
1−: The reason for using “1−” is to identify the proportion of the body that is not composed of fat. This allows us to isolate lean mass, which is more metabolically active and relevant for calculations such as calories burned at rest.

Further, formula to calculate the Daily Calories Needed (CDN)

### General Formula:

$$CDN = CQR \times Level of Activity$$

Activity Level (1.2 to 1.9): These coefficients adjust the CQR according to the level of daily physical activity. A lower value (1.2) represents a sedentary lifestyle, while a higher value (1.9) represents an extremely active lifestyle. These values are based on the amount of additional energy needed to maintain the corresponding level of physical activity.

Further, formula to determine the Percentage of Ideal Body Fat (PGI)

Adjusted PGI=PGI Base×(Exercise Frequency Factor−Muscle Mass Factor) Explanation of the Numbers:
PGI Base: It is the ideal percentage of body fat based on age and sex.
Exercise Frequency Factor (1.0 to 0.8): This adjustment reflects the frequency of exercise, decreasing the IGI in people who exercise more frequently, since they are likely to have less body fat.
Muscle Mass Factor (1.0 to 1.13): This factor adjusts the PGI according to the amount of muscle mass, since greater muscle mass is usually associated with a lower percentage of body fat.

Formula to calculate Ideal Body Fat (GCI) general formula:

$$GCI = PI \times ( Adjusted PGI - 100 ) Explanation of the Numbers :$$

PI (Ideal Weight): It is the optimal body weight previously calculated.

GCI=PI×(Adjusted PGI+100): Converts the percentage of adjusted ideal body fat into a decimal value, which is then multiplied by the ideal weight to obtain the ideal amount of body fat in kilograms.

Further, formula for calculating macronutrients based on the Daily Calories Needed (CDN) general formulas:

$$Carbohydrates ( g ) = ( CDN \times Percentage of Carbohydrates - 4 ) Proteins ( g ) = ( CDN \times Percentage of Proteins - 4 ) Fats ( g ) = ( CDN \times Percentage of Fats - 9 )$$

4 and 9: These numbers correspond to the amount of calories provided by each gram of carbohydrates or proteins (4 calories per gram) and fats (9 calories per gram). These values are used to convert the total calories into grams of each macronutrient.

Further, the percentages of macronutrients vary according to the objective (e.g., weight loss, muscle gain) and determine the proportion of calories that come from each macronutrient.

Further, the formula to distribute macronutrients in 3, 4, or 5 meals a day are as follows:

$$Carbohydrates per meal ( g ) = Carbohydrates ( g ) + Number of meals$$

$$Protein per meal ( g ) = Protein ( g ) + Number of meals$$

$$Fats per meal ( g ) = Fats ( g ) + Number of meals$$

Further, these calculations divide the daily intake of macronutrients into the number of meals the person wants to make in a day. This helps to plan meals in a balanced way and ensures that macronutrients are evenly distributed throughout the day.

Further, the formula for calculating the percentage of excess fat are as follows.

$$PGE = ( Current Weight ( kg ) - Ideal Weight ( kg ) ) + Current Weight ( kg ) \times 100$$

### Explanation of the Numbers:

(Current Weight-Ideal Weight): This calculation determines the amount of weight that is above the ideal weight, which usually represents excess body fat.

$$PGE = ( Current Weight ( kg ) - Ideal Weight ( kg ) ) and \times 100 :$$

Converting this difference into a percentage of the current total weight, providing a measure of excess body fat.

Further, the formula to calculate Daily Water Intake (IDA) General Formula is as follows.

IDA=(Current Weight (kg))×Adjustments)×0.035. Further, the number 0.035: represents 35 ml of water per kilogram of body weight, a generally accepted standard for maintaining adequate hydration.

Further, adjustments may include factors such as muscle mass, somatotype, age, lifestyle, stress level, hydration level and medical conditions, to customize water intake according to individual needs.

Further, each number and adjustment in these formulas has been carefully selected to reflect key aspects of human physiology and individual energy needs. These values ensure that the formulas are accurate, adaptable and useful for a wide range of people, facilitating health planning and fitness.

In some embodiments, the present disclosure describes a system designed to calculate an individual's personalized ideal weight based on various factors such as height, muscle mass, age, sex, and lifestyle. This core feature allows users to estimate their optimal body weight for maintaining good health and physical performance. The system employs complex algorithms that consider these factors, along with adjustment mechanisms to refine the calculation according to unique characteristics such as muscle mass and somatotype.

In some embodiments, the system may utilize AI-driven dynamic adjustments that adapt the ideal weight calculation based on real-time changes in lifestyle or environmental factors. These adjustments can be implemented through machine learning models that continuously monitor and update the user's data, providing a more responsive and accurate estimation of their optimal body weight.

Furthermore, the invention may incorporate a continuous health monitoring subsystem that tracks physiological indicators such as heart rate and blood pressure to refine the calculation. This system may use biofeedback techniques to adjust the ideal weight estimate based on the user's current health status, ensuring a more comprehensive approach to weight management.

Further, the system may integrate advanced visualization tools that present the calculated ideal weight in an intuitive format, such as comparative bar graphs or interactive dashboards. These tools can help users better understand their body composition and how it aligns with their health goals.

In some embodiments, the invention may include a feature that assesses the user's exercise routine and dietary habits to provide personalized recommendations that complement the calculated ideal weight. For instance, the system could recommend specific exercises or meal plans based on the individual's lifestyle and preferences, enhancing the effectiveness of the weight management strategy.

In some embodiments, the system may utilize wearable sensors to collect data on physical activity levels, sleep patterns, and other factors that influence body composition. These sensors can transmit data to the system, which then applies advanced analytics to determine the optimal weight range for the user, considering both static and dynamic variables.

Further, one technical problem addressed by these embodiments is the variability of factors influencing body weight, such as muscle mass fluctuations and changing lifestyle habits. The AI-driven adjustments and continuous monitoring help maintain the accuracy and relevance of the ideal weight estimate over time.

Further, in terms of implementation, the system may use a combination of machine learning algorithms and data analysis techniques to process input variables and generate tailored results. For example, the system might employ regression models that consider muscle mass as a key factor in adjusting the ideal weight calculation, ensuring that individuals with higher muscle mass receive more accurate estimates.

In some embodiments, the present disclosure may describe features that could involve a database of established formulas for lean body mass estimation, which the system selects or modifies based on the user's specific characteristics. This allows for dynamic adaptation of the calculation to different somatotypes and lifestyles.

In some embodiments, the integration of bioelectrical impedance devices is another example where the system may use these sensors to measure body composition metrics, providing more precise lean body mass data that informs the ideal weight calculation. This approach enhances the accuracy of calorie burn rate estimations, enabling users to make more informed dietary and exercise decisions.

Further, in some embodiments the present disclosure describes technical features and improvements aiming to enhance the personalization, adaptability, and practicality of the ideal weight calculation system, addressing various aspects of health and fitness estimation with innovative solutions that go beyond conventional methods.

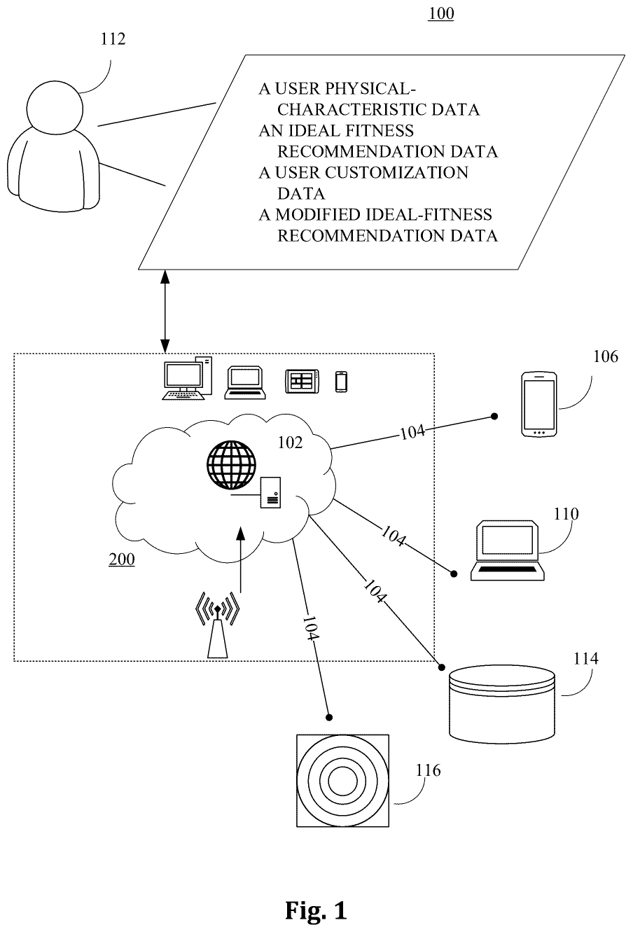

FIG. 1 is an illustration of an online platform 100 consistent with various embodiments of the present disclosure. By way of non-limiting example, the online platform 100 may be hosted on a centralized server 102, such as, for example, a cloud computing service. The centralized server 102 may communicate with other network entities, such as, for example, a mobile device 106 (such as a smartphone, a laptop, a tablet computer etc.), other electronic devices 110 (such as desktop computers, server computers etc.), databases 114, and sensors 116 over a communication network 104, such as, but not limited to, the Internet. Further, users of the online platform 100 may include relevant parties such as, but not limited to, end-users, administrators, service providers, service consumers and so on. Accordingly, in some instances, electronic devices operated by the one or more relevant parties may be in communication with the platform.

A user 112, such as the one or more relevant parties, may access online platform 100 through a web based software application or browser. The web based software application may be embodied as, for example, but not be limited to, a website, a web application, a desktop application, and a mobile application compatible with a computing device 200.

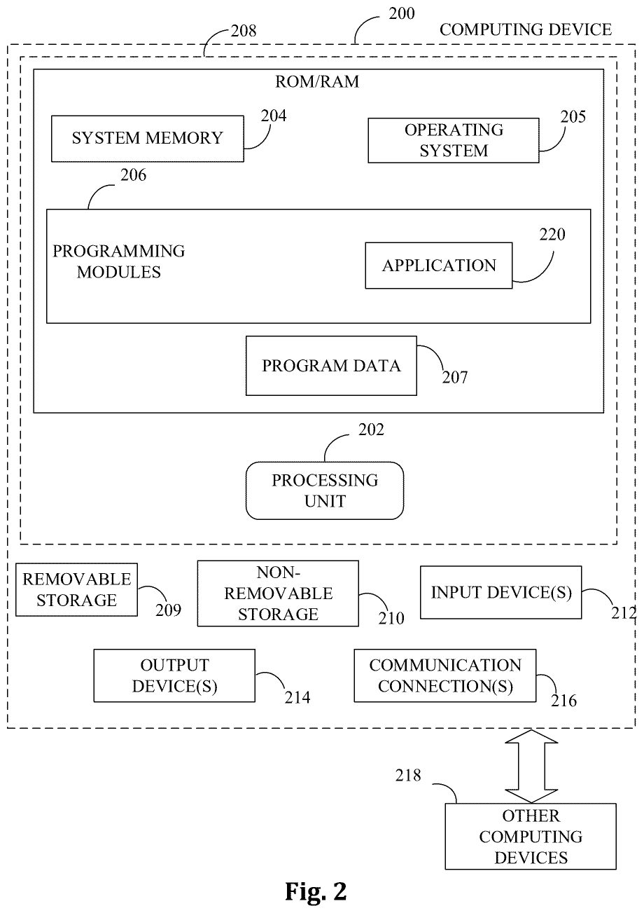

With reference to FIG. 2, a system consistent with an embodiment of the disclosure may include a computing device or cloud service, such as computing device 200. In a basic configuration, computing device 200 may include at least one processing unit 202 and a system memory 204. Depending on the configuration and type of computing device, system memory 204 may comprise, but is not limited to, volatile (e.g. random-access memory (RAM)), non-volatile (e.g. read-only memory (ROM)), flash memory, or any combination. System memory 204 may include operating system 205, one or more programming modules 206, and may include a program data 207. Operating system 205, for example, may be suitable for controlling computing device 200's operation. In one embodiment, programming modules 206 may include image-processing module, machine learning module. Furthermore, embodiments of the disclosure may be practiced in conjunction with a graphics library, other operating systems, or any other application program and is not limited to any particular application or system. This basic configuration is illustrated in FIG. 2 by those components within a dashed line 208.

Computing device 200 may have additional features or functionality. For example, computing device 200 may also include additional data storage devices (removable and/or non-removable) such as, for example, magnetic disks, optical disks, or tape. Such additional storage is illustrated in FIG. 2 by a removable storage 209 and a non-removable storage 210. Computer storage media may include volatile and non-volatile, removable and non-removable media implemented in any method or technology for storage of information, such as computer-readable instructions, data structures, program modules, or other data. System memory 204, removable storage 209, and non-removable storage 210 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 200. Any such computer storage media may be part of device 200. Computing device 200 may also have input device(s) 212 such as a keyboard, a mouse, a pen, a sound input device, a touch input device, a location sensor, a camera, a biometric sensor, etc. Output device(s) 214 such as a display, speakers, a printer, etc. may also be included. The aforementioned devices are examples and others may be used.

Computing device 200 may also contain a communication connection 216 that may allow device 200 to communicate with other computing devices 218, such as over a network in a distributed computing environment, for example, an intranet or the Internet. Communication connection 216 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 204, including operating system 205. While executing on processing unit 202, programming modules 206 (e.g., application 220 such as a media player) may perform processes including, for example, one or more stages of methods, algorithms, systems, applications, servers, databases as described above. The aforementioned process is an example, and processing unit 202 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

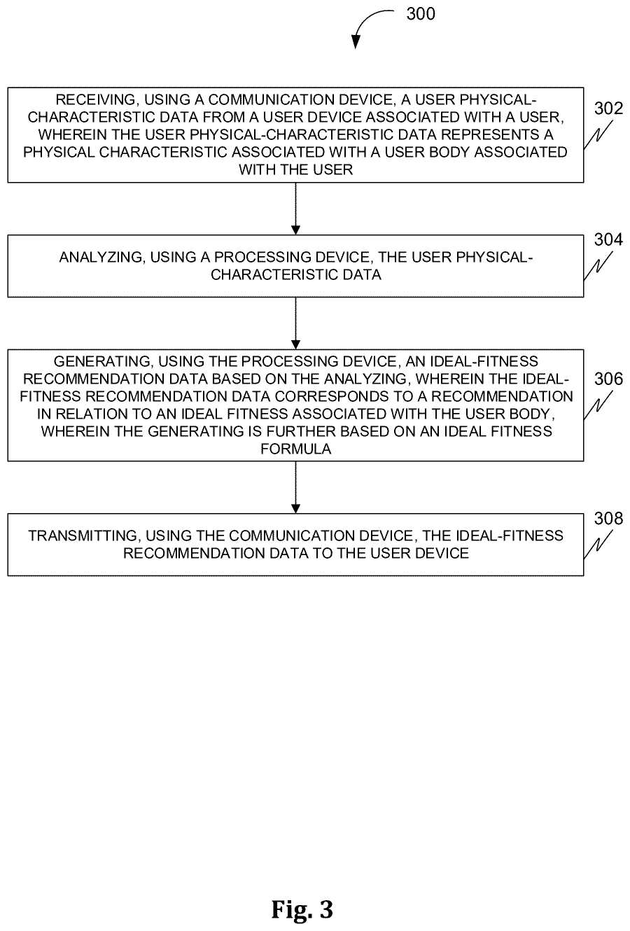

FIG. 3 illustrates a flowchart of a method 300 of provisioning a fitness recommendation in relation to a user, in accordance with some embodiments.

Accordingly, the method 300 may include a step 302 of receiving, using a communication device 502, user physical-characteristic data from a user device associated with a user. Further, the user physical-characteristic data represents a physical characteristic associated with a user body associated with the user. Further, the method 300 may include a step 304 of analyzing, using a processing device 504, the user physical-characteristic data. Further, the method 300 may include a step 306 of generating, using the processing device 504, ideal-fitness recommendation data based on the analyzing. Further, the ideal-fitness recommendation data corresponds to a recommendation in relation to an ideal fitness associated with the user body. Further, the generating may be further based on an ideal fitness formula. Further, the method 300 may include a step 308 of transmitting, using the communication device 502, the ideal-fitness recommendation data to the user device.

In some embodiments, the user physical-characteristic data includes gender selection data corresponding to a selection of a gender in relation to the user. Further, the method 300 further comprising determining, using the processing device 504, a gender-based attribute based on the gender selection data. Further, the gender-based attribute corresponds to an attribute based on the gender. Further, the generating of the ideal-fitness recommendation data may be further based on the determining of the gender-based attribute. Further, the gender-based attribute includes one or more of a body composition, a muscle mass, a bone density, a hormonal level and a body fat in relation to the user body.

In some embodiments, the user physical-characteristic data includes one or more of a user height data, a user weight data, a user calorie intake data and a physical activity level data associated with the user. Further, the user height data represents a height associated with the user. Further, the user weight data corresponds to a weight associated with the user. Further, the user calorie intake data corresponds to a calorie intake associated with the user. Further, the physical activity level data corresponds to a level associated with a physical activity performed by the user.

In some embodiments, the user physical-characteristic data includes a body-type data representing a body type associated with the user. Further, the method 300 further comprising determining, using the processing device 504, a distribution of each of the muscle mass and the body fat in relation to the user body based on the body-type data. Further, the generating of the ideal-fitness recommendation may be further based on the determining of the distribution. Further, the body type includes one or more of an ectomorph, a mesomorph and an endomorph. Further, the ectomorph may be characterized by a thin structure and a reduced muscle mass. Further, the mesomorph may be characterized by a muscular structure and a balanced distribution in relation to each of the muscle mass and body fat. Further, the endomorph may be characterized by a round-body structure and an accumulative fat distribution.

In some embodiments, the user physical-characteristic data includes a fitness objective data corresponding to a fitness objective in relation to the user body. Further, the fitness objective includes one or more of a weight loss, a weight gain, a weight retention and an elevation of muscle mass.

In some embodiments, the user physical-characteristic data includes a meal plan selection data corresponding to a selection in relation to a meal plan associated with the user. Further, the method 300 further comprising generating, using the processing device 504, a nutrient recommendation data based on the meal plan selection data. Further, the nutrient recommendation data corresponds to the recommendation in relation to a nutrient intake of the user. Further, the nutrient recommendation data may be comprised in the ideal-fitness recommendation data.

In some embodiments, the method 300 may further include generating, using the processing device 504, a calorific burn target data corresponding to a burn target in relation to the calorie associated with the user body. Further, the calorific burn target data may be comprised in the ideal-fitness recommendation data. Further, the user physical-characteristic data further includes a weight target data corresponding to a weight target in relation to the user body. Further, the ideal fitness formula includes a FCLP formula which may be configured to facilitate the generating of the calorific burn target data.

In some embodiments, the ideal-fitness recommendation data may be configured to be presented on a user presentation device associated with the user device. Further, the user device includes a user input device which may be configured for receiving a user customization data corresponding to a user customization in relation to the ideal-fitness recommendation data. Further, the user device further includes a user communication device which may be configured for transmitting the user customization data to the communication device 502.

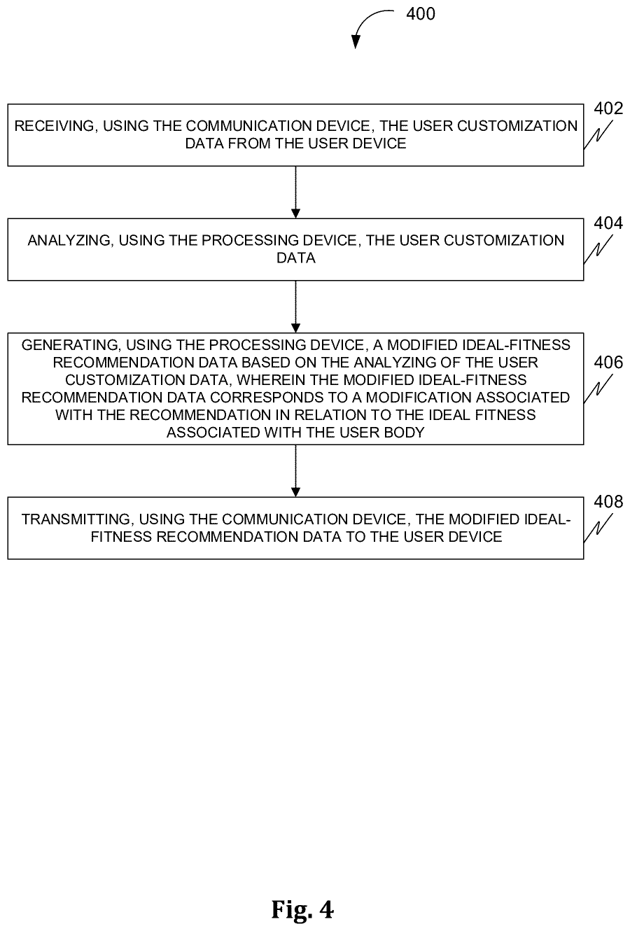

FIG. 4 illustrates a flowchart of a method 400 of provisioning a fitness recommendation in relation to a user including generating, using the processing device 504, a modified ideal-fitness recommendation data, in accordance with some embodiments.

Further, in some embodiments, the method 400 further may include a step 402 of receiving, using the communication device 502, the user customization data from the user device. Further, in some embodiments, the method 400 further may include a step 404 of analyzing, using the processing device 504, the user customization data. Further, in some embodiments, the method 400 further may include a step 406 of generating, using the processing device 504, a modified ideal-fitness recommendation data based on the analyzing of the user customization data. Further, the modified ideal-fitness recommendation data corresponds to a modification associated with the recommendation in relation to the ideal fitness associated with the user body. Further, in some embodiments, the method 400 further may include a step 408 of transmitting, using the communication device 502, the modified ideal-fitness recommendation data to the user device.

In some embodiments, the method 300 may further include generating, using the processing device 504, an ideal weight data based on the analyzing of the user physical-characteristic data. Further, the ideal weight data may be comprised in the ideal-fitness recommendation data. Further, the ideal fitness formula includes a FPI formula which may be configured for facilitating the generating of the ideal weight data.

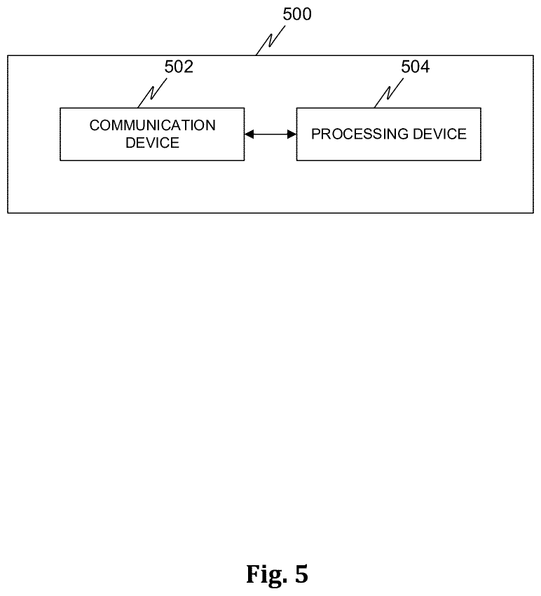

FIG. 5 illustrates a block diagram of a system 500 of provisioning a fitness recommendation in relation to a user, in accordance with some embodiments.

Accordingly, the system 500 may include a communication device 502. Further, the communication device 502 may be configured for receiving user physical-characteristic data from a user device associated with a user. Further, the user physical-characteristic data represents a physical characteristic associated with a user body associated with the user. Further, the communication device 502 may be configured for transmitting ideal-fitness recommendation data to the user device. Further, the system 500 may include a processing device 504. Further, the processing device 504 may be configured for analyzing the user physical-characteristic data. Further, the processing device 504 may be configured for generating the ideal-fitness recommendation data based on the analyzing. Further, the ideal-fitness recommendation data corresponds to a recommendation in relation to an ideal fitness associated with the user body. Further, the generating may be further based on an ideal fitness formula.

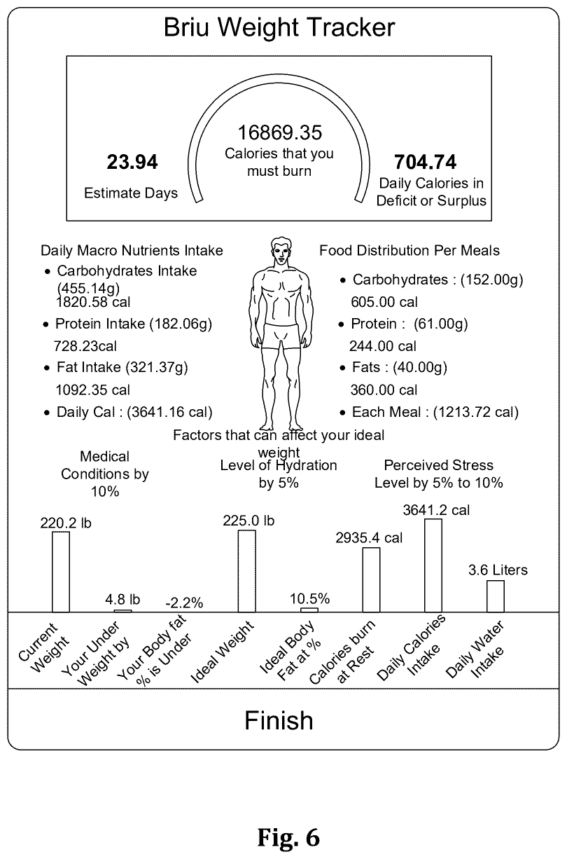

FIG. 6 illustrates a user interface of an application associated with systems and methods of provisioning a fitness recommendation in relation to a user, in accordance with some embodiments.

Further, in some embodiments the user interface shows the final summary screen in the “Briu Weight Tracker” application, which offers a complete breakdown of the user's fitness goals and personalized recommendations based on their data. The screen is divided into several sections that detail the caloric intake, the distribution of macronutrients, the factors that affect the ideal weight, and graphs that visualize the user's progress.

In some embodiments, the user physical-characteristic data includes gender selection data corresponding to a selection of a gender in relation to the user. Further, the system 500 further comprising determining, using the processing device 504, a gender-based attribute based on the gender selection data. Further, the gender-based attribute corresponds to an attribute based on the gender. Further, the generating of the ideal-fitness recommendation data may be further based on the determining of the gender-based attribute. Further, the gender-based attribute includes one or more of a body composition, a muscle mass, a bone density, a hormonal level and a body fat in relation to the user body.

In some embodiments, the user physical-characteristic data includes one or more of a user height data, a user weight data, a user calorie intake data and a physical activity level data associated with the user. Further, the user height data represents a height associated with the user. Further, the user weight data corresponds to a weight associated with the user. Further, the user calorie intake data corresponds to a calorie intake associated with the user. Further, the physical activity level data corresponds to a level associated with a physical activity performed by the user.

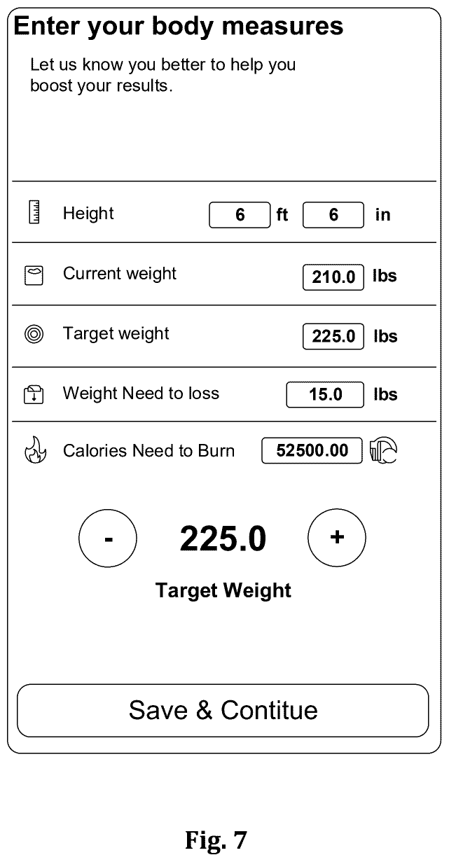

FIG. 7 illustrates a user interface of an application associated with systems and methods of provisioning a fitness recommendation in relation to a user, in accordance with some embodiments.

Further, in some embodiments the user interface shows the body measurement input screen in the “Briu Fitness App” (or “Briu Fitness Studio App”) application. The interface is designed to collect basic user information, such as height, current weight, target weight, and calories the user needs to burn, to customize the results and optimize the performance of the fitness program.

In some embodiments, the user physical-characteristic data includes a body-type data representing a body type associated with the user. Further, the processing device 504 may be further configured for determining a distribution of each of the muscle mass and the body fat in relation to the user body based on the body-type data. Further, the generating of the ideal-fitness recommendation may be further based on the determining of the distribution. Further, the body type includes one or more of an ectomorph, a mesomorph and an endomorph. Further, the ectomorph may be characterized by a thin structure and a reduced muscle mass. Further, the mesomorph may be characterized by a muscular structure and a balanced distribution in relation to each of the muscle mass and body fat. Further, the endomorph may be characterized by a round-body structure and an accumulative fat distribution.

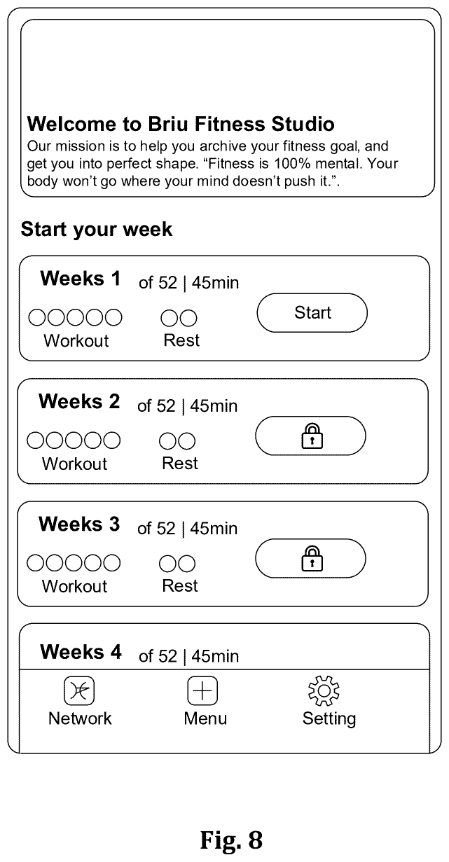

FIG. 8 illustrates a user interface of an application associated with systems and methods of provisioning a fitness recommendation in relation to a user, in accordance with some embodiments.

Further, in some embodiments the user interface shows the start of the week screen in the “Briu Fitness App” (or “Briu Fitness Studio App”) application, where the user may manage and follow their weekly training program. The interface is designed to guide the user through a fitness plan structured by weeks, with details about training sessions and rest days.

In some embodiments, the user physical-characteristic data includes a fitness objective data corresponding to a fitness objective in relation to the user body. Further, the fitness objective includes one or more of a weight loss, a weight gain, a weight retention and an elevation of muscle mass.

In some embodiments, the user physical-characteristic data includes a meal plan selection data corresponding to a selection in relation to a meal plan associated with the user. Further, the processing device 504 may be further configured for generating a nutrient recommendation data based on the meal plan selection data. Further, the nutrient recommendation data corresponds to the recommendation in relation to a nutrient intake of the user. Further, the nutrient recommendation data may be comprised in the ideal-fitness recommendation data.

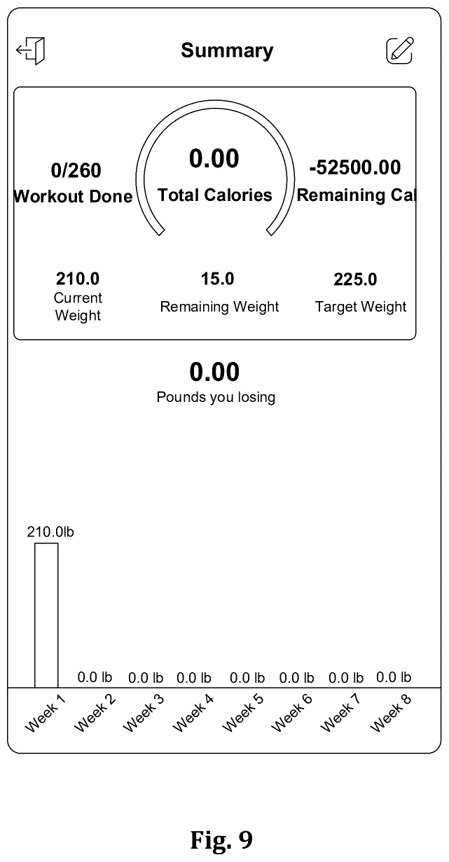

FIG. 9 illustrates a user interface of an application associated with systems and methods of provisioning a fitness recommendation in relation to a user, in accordance with some embodiments.

Further, in some embodiments the user interface may include the summary screen in the “Briu Fitness App” application (or “Briu Fitness Studio App”), where the user may view a summary of their progress in the fitness program. The interface is designed to provide an overview of the calories burned, the current weight, the target weight and the overall progress in terms of weight loss.

In some embodiments, the processing device 504 id further which may be configured for generating a calorific burn target data corresponding to a burn target in relation to the calorie associated with the user body. Further, the calorific burn target data may be comprised in the ideal-fitness recommendation data. Further, the user physical-characteristic data further includes a weight target data corresponding to a weight target in relation to the user body. Further, the ideal fitness formula includes a FCLP formula which may be configured to facilitate the generating of the calorific burn target data.

In some embodiments, the ideal-fitness recommendation data may be configured to be presented on a user presentation device associated with the user device. Further, the user device includes a user input device which may be configured for receiving a user customization data corresponding to a user customization in relation to the ideal-fitness recommendation data. Further, the user device further includes a user communication device which may be configured for transmitting the user customization data to the communication device 502.

Further, in some embodiments, the communication device 502 may be further configured for receiving the user customization data from the user device. Further, the communication device 502 may be further configured for transmitting a modified ideal-fitness recommendation data to the user device. Further, the processing device 504 may be further configured for. Further, the communication device 502 may be further configured for analyzing the user customization data. Further, the communication device 502 may be further configured for generating the modified ideal-fitness recommendation data based on the analyzing of the user customization data. Further, the modified ideal-fitness recommendation data corresponds to a modification associated with the recommendation in relation to the ideal fitness associated with the user body.

In some embodiments, the processing device 504 may be further configured for generating an ideal weight data based on the analyzing of the user physical-characteristic data. Further, the ideal weight data may be comprised in the ideal-fitness recommendation data. Further, the ideal fitness formula includes a FPI formula which may be configured for facilitating the generating of the ideal weight data.

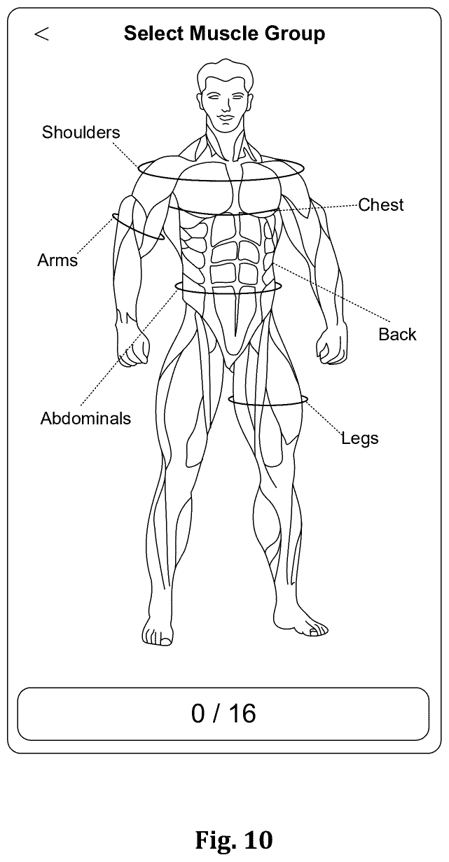

FIG. 10 illustrates a user interface of an application associated with systems and methods of provisioning a fitness recommendation in relation to a user, in accordance with some embodiments.

Further, in some embodiments the user interface may include the “Select Muscle Group” screen in the “Briu Fitness App” (or “Briu Fitness Studio App”) application. The interface is designed for the user to select the muscle groups they want to focus on during their training session.

In some embodiments, the gender includes one or more of a male gender and a female gender.

In some embodiments, the gender-based attribute in relation to the male gender includes one or more of an elevated muscle mass and an average bone density.

In some embodiments, the gender-based attribute in relation to the female gender includes one or more of an elevated fat percentage and a hormonal difference. Further, the hormonal difference may be in relation to the male gender.

In some embodiments, the user physical-characteristic data includes a user age data corresponding to an age associated with the user. Further, the method 300 further comprising determining, using the processing device 504, a state associated with each of the body composition and a metabolism associated with the user body based on the user age data. Further, the generating of the ideal-fitness recommendation data may be further based on the state.

In some embodiments, the user age includes one or more of a young age and an old age. Further, the young age may be characterized by a state of an elevated muscle mass and a faster metabolism. Further, the old age may be characterized by a reduced muscle mass, a reduced bone density and a slower metabolism.

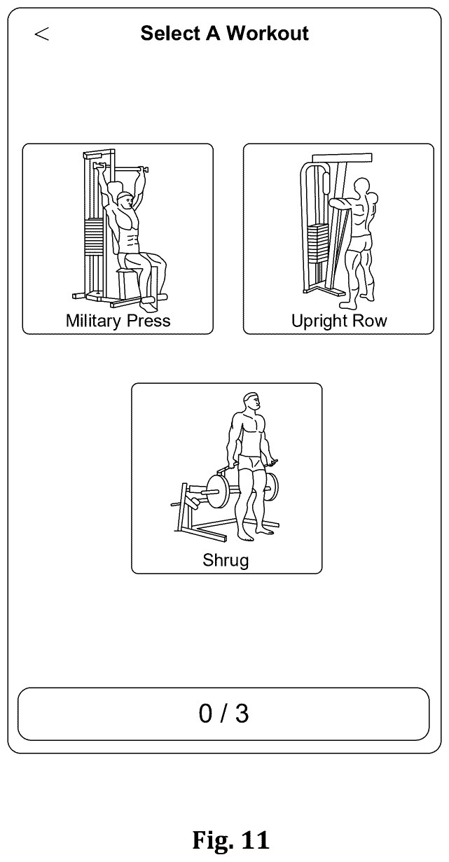

FIG. 11 illustrates a user interface of an application associated with systems and methods of provisioning a fitness recommendation in relation to a user, in accordance with some embodiments.

Further, in some embodiments the user interface may include the “Select A Workout” screen in the “Briu Fitness App” (or “Briu Fitness Studio App”) application. This interface allows the user to select between different specific exercises that they want to perform during their training session.

In some embodiments, the determining of the distribution of each of the muscle mass and the body fat in relation to the user body may be further based on the user height associated with the user.

In some embodiments, the meal plan includes one or more of a three meal a day plan, a four meal a day plan and a five meal a day plan associated with the user.

In some embodiments, the nutrient recommendation data includes a nutrient distribution data corresponding to a distribution of the nutrition intake associated with the user. Further, the generating of the nutrition distribution data may be based on meal plan selection data.

In some embodiments, the nutrition intake includes a macronutrient intake. Further, the macronutrient intake includes one or more of a protein intake, a carbohydrate intake and a fat intake associated with the user.

In some embodiments, the physical activity level data includes a physical activity frequency data corresponding to a frequency associated with the physical activity performed by the user. Further, the frequency may be associated with a duration. Further, the duration includes a weekly duration.

In some embodiments, the frequency includes one or more of a nil frequency, a one to two times per week frequency, a three to four times per week frequency, a five to six times per week frequency and a seven times per week frequency.

In some embodiments, the generating of the nutrition recommendation data may be further based on the physical activity frequency data.

In some embodiments, the nutrition recommendation data includes a minimum calorie intake recommendation data corresponding to the recommendation for a minimum calorie intake in relation to the nil frequency.

In some embodiments, the nutrition recommendation data includes a mild calorie intake recommendation data corresponding to the recommendation for a mild calorie intake in relation to the one to two times per week frequency.

In some embodiments, the nutrition recommendation data includes a moderate calorie intake recommendation data corresponding to the recommendation for a moderate calorie intake in relation to the three to four times per week frequency.

In some embodiments, the nutrition recommendation data includes a high calorie intake recommendation data corresponding to the recommendation for a high calorie intake in relation to the five to six times per week frequency.

In some embodiments, the nutrition recommendation data includes a maximum calorie intake recommendation data corresponding to the recommendation for a maximum calorie intake in relation to the seven times per week frequency.

In some embodiments, the ideal-fitness recommendation data includes a fitness impacting-factor data corresponding to a factor affecting the ideal fitness associated with the user body.

In some embodiments, the ideal-fitness recommendation data further includes a fitness routine data corresponding to a routine in relation to the ideal fitness associated with the user body.

In some embodiments, the ideal-fitness recommendation data further includes a graphical representation data corresponding to a graphical representation of a progress associated with the user in relation to the routine.

In some embodiments, the user physical-characteristic data includes one or more of a medical condition data, a hydration level data and a stress level data associated with the user.

In some embodiments, the fitness routine data includes a structured fitness plan data corresponding to a structured plan in relation to ideal fitness associated with the user. Further, the structured plan may be associated with a plan duration corresponding to a duration in relation to the structured plan.

In some embodiments, the structured fitness plan data includes a weekly fitness plan data corresponding to the structured plan associated with a week.

In some embodiments, the weekly fitness plan data includes two or more weekly fitness plan data associated with two or more weeks. Further, the two or more weekly fitness plan data includes a first week fitness plan data associated with a first week and a second week fitness plan data associated with a second week.

In some embodiments, the method 300 may further include generating, using the processing device 504, a user engagement data based on a user engagement in relation to the ideal-fitness recommendation data. Further, the user engagement data includes one or more of a first week user engagement data and second week user engagement data. Further, the first week user engagement data corresponds to the user engagement in relation to the first week fitness plan data. Further, the second week user engagement data corresponds to the user engagement in relation to the second week fitness plan data.

In some embodiments, the method 300 may further include analyzing, using the processing device 504, each of the first week user engagement data and the second week user engagement data. Further, the transmitting of the second week fitness plan data may be based on the analyzing of the first week user engagement data associated with the user.

In some embodiments, the method 300 may further include generating, using the processing device 504, each of a muscle growth estimation data and a progressive weight loss data. Further, the muscle growth estimation data corresponds to an estimation in relation to a muscle growth associated with the user body. Further, the progressive weight loss data corresponds to a progressive weight loss schedule in relation to the user body. Further, each of the muscle growth estimation data and the progressive weight loss data may be comprised in the ideal-fitness recommendation data.

In some embodiments, the ideal fitness formula includes a FCMP formula which may be configured for facilitating the generating of each of the muscle growth estimation data and the progressive weight loss data.

In some embodiments, the ideal-fitness recommendation data includes a user water-intake data representing a water-intake progress in relation to the user.

In some embodiments, the user water-intake data includes one or more of a daily water intake data and an annual water intake data. Further, the daily water intake data corresponds to the water-intake progress associated with the user in a day. Further, the annual water intake data corresponds to the water-intake progress associated with the user in a year.

In some embodiments, the ideal-fitness recommendation data includes a workout recommendation data corresponding to a recommendation in relation to a workout routine associated with the user.

In some embodiments, the ideal-fitness recommendation data includes a muscle report data corresponding to a report in relation to two or more muscle areas associated with the user body. Further, the muscle report data further corresponds to each of the muscle areas targeted in relation to the workout routine.

In some embodiments, the user customization data includes a workout customization data corresponding to the user customization in relation to the workout routine associated with the user.

In some embodiments, the muscle report data includes a muscle performance data representing a performance analysis of each of the two or more muscle areas associated with the user. Further, the performance analysis may be in relation to the workout routine associated with the user.

In some embodiments, the muscle performance data includes one or more of a muscle gain data, a calorie burn data and a fat loss data associated with the user.

In some embodiments, the ideal-fitness recommendation data includes a weekly performance data corresponding to performance analysis of the user in relation to the workout routine. Further, the performance analysis may be associated with a duration.

Further, the duration includes a weekly duration.

In some embodiments, the user customization data includes a muscle area selection data corresponding to a user selection in relation to the two or more muscle areas.

In some embodiments, the workout recommendation data includes a military press recommendation data corresponding to the recommendation in relation to a military press routine associated with the user.

In some embodiments, the workout recommendation data includes a chest press recommendation data corresponding to the recommendation in relation to a chest press routine associated with the user.

In some embodiments, the workout recommendation data includes one or more of a weight recommendation data and a repetition recommendation data. Further, the weight recommendation data corresponds to the recommendation in relation to the weight associated with the workout routine. Further, the repetition recommendation data corresponds to the recommendation in relation to a repetition associated with the weight in the workout routine.

In some embodiments, the weight includes two or more weights. Further, the two or more weights includes one or more of a starting weight, an incremented weight and a target weight. Further, the weight recommendation data includes one or more of a starting weight recommendation data, an incremental weight recommendation data and a target weight recommendation data. Further, the starting weight recommendation data corresponds to the recommendation in relation to the starting weight which may be configured to start the workout routine. Further, the incremental weight recommendation data corresponds to the recommendation in relation to the incremented weight associated with the workout routine. Further, the target weight recommendation data corresponds to the recommendation in relation to the target weight targeted by the user in relation to the workout routine.

In some embodiments, the repetition includes a five repetition.

In some embodiments, the workout recommendation data may be further configured for enhancing one or more of a technique and a performance in relation to the user.

In some embodiments, the repetition may be associated with a repetition duration corresponding to a duration in relation to the repetition.

In some embodiments, the workout routine may be associated with a MET factor representing an intensity in relation to the workout routine.

In some embodiments, the method 300 may further include generating, using the processing device 504, a calorific burn target data corresponding to a burn target in relation to the calorie associated with the user body. Further, the calorific burn target data may be comprised in the ideal-fitness recommendation data. Further, the ideal fitness formula includes a FCLP formula which may be configured to facilitate the generating of the calorific burn target data.

In some embodiments, the calorific burn target data may be further generated by a product of each of the MET factor, the weight, a pound to kilogram conversion factor, a quantity of repetitions, an oxygen factor, the repetition duration and an intensity factor associated with the workout routine. Further, the product may be transformed into a value expressed in minutes by dividing by a factor of sixty.

In some embodiments, the pound to kilogram conversion factor includes a zero point four five three five nine two conversion factor. Further, the oxygen factor includes a zero point zero one seven five factor.

In some embodiments, the intensity factor may be associated with an intensity factor range corresponding to a range in relation to the intensity factor. Further, the range includes a one to one-point five range.

In some embodiments, the intensity factor includes one or more of a low intensity factor, a moderate intensity factor and a high intensity factor. Further, the intensity factor range in relation to the moderate intensity factor includes a one-point one to a one-point three range. Further, the intensity factor range in relation to the high intensity factor includes a one-point four to a one-point five range.

In some embodiments, the method 300 may further include generating, using the processing device 504, a daily training volume data representing a daily training volume associated with the user. Further, the generating of the ideal-fitness recommendation data may be further based on the daily training volume data. Further, the daily training volume corresponds to as a sum of each of the product of the starting weight and the repetitions in relation to the starting weight, the product of incremented weight and the repetitions in relation to the incremented weight and the product of target weight, the repetitions in relation to the target weight, and a quantity of the two or more weights in relation to the workout routine.

In some embodiments, the workout routine may be associated with a workout duration corresponding to a duration in relation to the workout routine. Further, the workout duration includes a weekly workout duration corresponding to the duration of the workout routine in a week. Further, the ideal-fitness recommendation data includes a weekly training volume data representing a weekly training volume associated with the user. Further, the weekly training volume corresponds to the product of each of the daily training volume and the weekly workout duration.

In some embodiments, the workout routine may be further associated with a proportionality factor which may be configured to relate the weekly training volume to a muscle growth in relation to the user body, may. Further, the ideal-fitness recommendation data includes a weekly muscle growth data representing a weekly muscle growth associated with the user body. Further, the weekly muscle growth corresponds to the product of each of the weekly training volume and the proportionality factor.

In some embodiments, the generating of the ideal weight data may be further based on determining a sum of a basic weight value and a product of an ideal weight increment with a difference obtained by subtracting the basic height value from the user height, and subsequently multiplying the sum by an adjustment value.

Although the invention has been explained in relation to its preferred embodiment, it is to be understood that many other possible modifications and variations can be made without departing from the spirit and scope of the invention as hereinafter claimed.

1. A method of provisioning a fitness recommendation in relation to a user, wherein the method comprising:
receiving, using a communication device, user physical-characteristic data from a user device associated with a user, wherein the user physical-characteristic data represents a physical characteristic associated with a user body associated with the user;
analyzing, using a processing device, the user physical-characteristic data;
generating, using the processing device, ideal-fitness recommendation data based on the analyzing, wherein the ideal-fitness recommendation data corresponds to a recommendation in relation to an ideal fitness associated with the user body, wherein the generating is further based on an ideal fitness formula; and
transmitting, using the communication device, the ideal-fitness recommendation data to the user device.

2. The method of claim 1, wherein the user physical-characteristic data comprises gender selection data corresponding to a selection of a gender in relation to the user, wherein the method further comprising determining, using the processing device, a gender-based attribute based on the gender selection data, wherein the gender-based attribute corresponds to an attribute based on the gender, wherein the generating of the ideal-fitness recommendation data is further based on the determining of the gender-based attribute, wherein the gender-based attribute comprises at least one of a body composition, a muscle mass, a bone density, a hormonal level and a body fat in relation to the user body.

3. The method of claim 2, wherein the user physical-characteristic data comprises at least one of a user height data, a user weight data, a user calorie intake data and a physical activity level data associated with the user, wherein the user height data represents a height associated with the user, wherein the user weight data corresponds to a weight associated with the user, wherein the user calorie intake data corresponds to a calorie intake associated with the user, wherein the physical activity level data corresponds to a level associated with a physical activity performed by the user.

4. The method of claim 3, wherein the user physical-characteristic data comprises a body-type data representing a body type associated with the user, wherein the method further comprising determining, using the processing device, a distribution of each of the muscle mass and the body fat in relation to the user body based on the body-type data, wherein the generating of the ideal-fitness recommendation is further based on the determining of the distribution, wherein the body type comprises at least one of an ectomorph, a mesomorph and an endomorph, wherein the ectomorph is characterized by a thin structure and a reduced muscle mass, wherein the mesomorph is characterized by a muscular structure and a balanced distribution in relation to each of the muscle mass and body fat, wherein the endomorph is characterized by a round-body structure and an accumulative fat distribution.

5. The method of claim 3, wherein the user physical-characteristic data comprises a fitness objective data corresponding to a fitness objective in relation to the user body, wherein the fitness objective comprises at least one of a weight loss, a weight gain, a weight retention and an elevation of muscle mass.

6. The method of claim 5, wherein the user physical-characteristic data comprises a meal plan selection data corresponding to a selection in relation to a meal plan associated with the user, wherein the method further comprising generating, using the processing device, a nutrient recommendation data based on the meal plan selection data, wherein the nutrient recommendation data corresponds to the recommendation in relation to a nutrient intake of the user, wherein the nutrient recommendation data is comprised in the ideal-fitness recommendation data.

7. The method of claim 1 further comprising generating, using the processing device, a calorific burn target data corresponding to a burn target in relation to the calorie associated with the user body, wherein the calorific burn target data is comprised in the ideal-fitness recommendation data, wherein the user physical-characteristic data further comprises a weight target data corresponding to a weight target in relation to the user body, wherein the ideal fitness formula comprises a FCLP formula configured to facilitate the generating of the calorific burn target data.

8. The method of claim 1, wherein the ideal-fitness recommendation data is configured to be presented on a user presentation device associated with the user device, wherein the user device comprises a user input device configured for receiving a user customization data corresponding to a user customization in relation to the ideal-fitness recommendation data, wherein the user device further comprises a user communication device configured for transmitting the user customization data to the communication device.

9. The method of claim 8 further comprising:
receiving, using the communication device, the user customization data from the user device;
analyzing, using the processing device, the user customization data;
generating, using the processing device, a modified ideal-fitness recommendation data based on the analyzing of the user customization data, wherein the modified ideal-fitness recommendation data corresponds to a modification associated with the recommendation in relation to the ideal fitness associated with the user body; and
transmitting, using the communication device, the modified ideal-fitness recommendation data to the user device.

10. The method of claim 1 further comprising generating, using the processing device, an ideal weight data based on the analyzing of the user physical-characteristic data, wherein the ideal weight data is comprised in the ideal-fitness recommendation data, wherein the ideal fitness formula comprises a FPI formula configured for facilitating the generating of the ideal weight data.

11. A system of provisioning a fitness recommendation in relation to a user, wherein the system comprising:
a communication device configured for:
receiving user physical-characteristic data from a user device associated with a user, wherein the user physical-characteristic data represents a physical characteristic associated with a user body associated with the user; and
transmitting ideal-fitness recommendation data to the user device; and
a processing device configured for:
analyzing the user physical-characteristic data; and
generating the ideal-fitness recommendation data based on the analyzing, wherein the ideal-fitness recommendation data corresponds to a recommendation in relation to an ideal fitness associated with the user body, wherein the generating is further based on an ideal fitness formula.

12. The system of claim 11, wherein the user physical-characteristic data comprises gender selection data corresponding to a selection of a gender in relation to the user, wherein the system further comprising determining, using the processing device, a gender-based attribute based on the gender selection data, wherein the gender-based attribute corresponds to an attribute based on the gender, wherein the generating of the ideal-fitness recommendation data is further based on the determining of the gender-based attribute, wherein the gender-based attribute comprises at least one of a body composition, a muscle mass, a bone density, a hormonal level and a body fat in relation to the user body.

13. The system of claim 12, wherein the user physical-characteristic data comprises at least one of a user height data, a user weight data, a user calorie intake data and a physical activity level data associated with the user, wherein the user height data represents a height associated with the user, wherein the user weight data corresponds to a weight associated with the user, wherein the user calorie intake data corresponds to a calorie intake associated with the user, wherein the physical activity level data corresponds to a level associated with a physical activity performed by the user.

14. The system of claim 13, wherein the user physical-characteristic data comprises a body-type data representing a body type associated with the user, wherein the processing device is further configured for determining a distribution of each of the muscle mass and the body fat in relation to the user body based on the body-type data, wherein the generating of the ideal-fitness recommendation is further based on the determining of the distribution, wherein the body type comprises at least one of an ectomorph, a mesomorph and an endomorph, wherein the ectomorph is characterized by a thin structure and a reduced muscle mass, wherein the mesomorph is characterized by a muscular structure and a balanced distribution in relation to each of the muscle mass and body fat, wherein the endomorph is characterized by a round-body structure and an accumulative fat distribution.

15. The system of claim 13, wherein the user physical-characteristic data comprises a fitness objective data corresponding to a fitness objective in relation to the user body, wherein the fitness objective comprises at least one of a weight loss, a weight gain, a weight retention and an elevation of muscle mass.

16. The system of claim 15, wherein the user physical-characteristic data comprises a meal plan selection data corresponding to a selection in relation to a meal plan associated with the user, wherein the processing device is further configured for generating a nutrient recommendation data based on the meal plan selection data, wherein the nutrient recommendation data corresponds to the recommendation in relation to a nutrient intake of the user, wherein the nutrient recommendation data is comprised in the ideal-fitness recommendation data.

17. The system of claim 11, wherein the processing device id further configured for generating a calorific burn target data corresponding to a burn target in relation to the calorie associated with the user body, wherein the calorific burn target data is comprised in the ideal-fitness recommendation data, wherein the user physical-characteristic data further comprises a weight target data corresponding to a weight target in relation to the user body, wherein the ideal fitness formula comprises a FCLP formula configured to facilitate the generating of the calorific burn target data.

18. The system of claim 11, wherein the ideal-fitness recommendation data is configured to be presented on a user presentation device associated with the user device, wherein the user device comprises a user input device configured for receiving a user customization data corresponding to a user customization in relation to the ideal-fitness recommendation data, wherein the user device further comprises a user communication device configured for transmitting the user customization data to the communication device.

19. The system of claim 18, wherein the communication device is further configured for:
receiving the user customization data from the user device; and
transmitting a modified ideal-fitness recommendation data to the user device, wherein the processing device is further configured for:
analyzing the user customization data; and
generating the modified ideal-fitness recommendation data based on the analyzing of the user customization data, wherein the modified ideal-fitness recommendation data corresponds to a modification associated with the recommendation in relation to the ideal fitness associated with the user body.

20. The system of claim 11, wherein the processing device is further configured for generating an ideal weight data based on the analyzing of the user physical-characteristic data, wherein the ideal weight data is comprised in the ideal-fitness recommendation data, wherein the ideal fitness formula comprises a FPI formula configured for facilitating the generating of the ideal weight data.
